(19) United States
(12) Patent Application Publication
(10) Pub. No.: US 2026/0224426 A1
(43) Pub. Date: Aug. 6, 2026

(54) Methods, Systems, and Apparatuses, for Initiating or Terminating Multi-Joint Assistance for Leg Movement

(71) Applicants: The United States Government as represented by the Department of Veterans Affairs, Washignton, DC (US); CASE WESTERN RESERVE UNIVERSITY, Cleveland, OH (US)

(72) Inventors: Nathaniel S. Makowski, Cleveland, OH (US); Lisa M. Lombardo, Chardon, OH (US); Hailey Heidecker, Cleveland Heights, OH (US); Ronald J. Triolo, Cleveland Heights, OH (US); Marshaun N. Fitzpatrick, Oberlin, OH (US)

(21) Appl. No.: 19/119,330

(22) PCT Filed: Oct. 16, 2023
(86) PCT No.: PCT/US2023/076963

§ 371 Date: Apr. 8, 2025

Related U.S. Application Data
(60) Provisional application No. 63/416,303, filed on Oct. 14, 2022.

(51) Int. Cl. A61H 3/00; A61H 1/02; A61N 1/36

(52) U.S. Cl. CPC A61H 3/00; A61H 1/024; A61H 2003/007; A61H 2201/10; A61H 2201/1207; A61H 2201/5007; A61H 2230/625; A61N 1/36003

(57) ABSTRACT

Methods, systems, and/or apparatuses are provided for providing and/or terminating multi-joint assistance for leg movement of a user. The assistance may be provided to a paretic leg or prosthetic leg of the user. Kinematic data associated with the paretic or prosthetic leg of the user may be received. A current phase of a gait motion for the paretic or prosthetic leg may be determined and/or the kinematic data may be compared to one or more thresholds to determine if thresholds have been satisfied. One or more of electrical stimuli or motorized assistance may be provided to the paretic or prosthetic leg or the supply may be terminated or reduced based on the current phase of the gait motion of the paretic or prosthetic leg and/or the one or more thresholds being satisfied.

### CROSS-REFERENCE TO RELATED APPLICATIONS

This Application claims priority to and the benefit of U.S. Provisional Patent Application No. 63/416,303, filed Oct. 14, 2022, the entire contents of which are hereby incorporated herein by reference in its entirety.

### BACKGROUND

Stroke survivors as well as those with other ailments may suffer from partial paralysis or reduced capabilities in one of their legs (e.g., a paretic leg). These people may have difficulty walking or relearning to walk without some sort of assistance. Providing motorized assistance or electrical stimulation to portions of the paretic leg through different portions of the gait cycle has the potential to substantially improve walking. Conventional devices may provide motorized assistance during all or certain portions of the gait cycle. However, these conventional devices can be difficult to operate as they lack the ability to recognize, within the gait cycle, when a person may be initiating a gait cycle or stopping a gait cycle in order to stand still. This can cause these conventional devices to not provide the proper assistance when the person begins the gait cycle. This can also cause these conventional devices to continue providing assistance, via motorized assistance or electrical stimulus, even when the person is trying to stop walking (e.g., stop a gait cycle). This can cause frustration with the person and can further increase the potential of causing injury to the person based on the unexpected assistance, or lack thereof, provided by these conventional devices.

### SUMMARY

Described herein, in various aspects, are methods, systems, and apparatuses configured to provide multi-joint assistance for walking. For example, multi-joint assistance may be provided by an apparatus that is removably coupled to a leg of a person (i.e., patient or user). For example, the leg may be a paretic leg of the user who has previously suffered a stroke or other ailment.

In certain examples, the apparatus may comprise a brace. The brace may comprise a thigh section, a shank section, and a foot support section. The thigh section and the shank section may be movably coupled to one another and the shank section and the foot support section may be movably coupled to one another. The brace may comprise a motor configured to provide motorized assistance between the thigh section and the shank section. The brace may comprise one or more electrodes and an electrical power source electrically coupled thereto. The one or more electrodes may be configured to provide electrical stimuli to a thigh portion and/or shank portion of the paretic leg of the user. The brace may comprise one or more sensors configured to determine kinematic information associated with the leg or a portion of the paretic leg. For example, the brace may comprise one or more of a thigh sensor, a shank sensor, or a heel strike sensor. For example, heel strike sensors may be provided for both the foot of the paretic leg and the foot of the non-paretic leg of the user. The brace may comprise one or more devices or mechanisms for attaching the brace to the paretic leg of the user. The apparatus may comprise a control computing device. The control computing device may be a computer configured to receive sensor data from the one or more sensors and determine to initiate or terminate one or more of the motorized assistance or the electrical stimuli to all or a portion of the paretic leg.

In certain examples, a method for providing assistance to a paretic leg may be provided. The method may comprise receiving kinematic data associated with a paretic leg. The kinematic data may be received by a computing device, such as the control computing device. A current phase of the gait motion of the paretic leg may be determined. For example, the current phase may be determined based on the kinematic data. One or both of electrical stimuli or motorized assistance may be provided by the brace to the paretic leg. For example, the electrical stimuli and/or the motorized assistance may be provided based on the current phase of the gait motion of the paretic leg.

In certain examples, a method for providing assistance to a paretic leg may be provided. The method may comprise receiving kinematic data for a portion of a paretic leg of a user. The method may comprise determining an orientation of the portion of the paretic leg satisfies a threshold. The determination may be based on the kinematic data. The method may comprise initiating at least one of electrical stimuli or motorized assistance for at least the portion of the paretic leg. For example, initiating at least one of electrical stimuli or motorized assistance may be based on the orientation of the paretic leg satisfying the threshold.

In certain examples, a method for terminating assistance to a paretic leg may be provided. The method may comprise receiving first kinematic data or heel-strike data from a portion of a non-paretic leg of a user. The first kinematic data and/or heel-strike data may be indicative of foot-floor contact (e.g., the foot contacting the floor) for a portion of the non-paretic leg of the user. The method may comprise receiving second kinematic data for a portion of a paretic leg of the user. The method may comprise determining an orientation of the portion of the paretic leg satisfies a threshold. For example, the determination may be based on the second kinematic data. The method may comprise terminating or reducing at least one of a supply of electrical stimuli or motorized assistance to at least the portion of the paretic leg of the user. For example, terminating or reducing the electrical supply and/or motorized assistance may be based on the first kinematic data or the heel-strike data indicating foot-floor contact for the portion of the non-paretic leg and the orientation of the portion of the paretic leg satisfying the threshold.

In certain examples, a method for terminating or reducing assistance to a paretic leg may be provided. The method may comprise receiving kinematic data for a portion of a paretic leg of a user. For example, the kinematic data may comprise an angular velocity for the portion of the paretic leg and an orientation for the portion of the paretic leg. The method may comprise determining the angular velocity satisfies an angular velocity threshold. The method may comprise determining the orientation satisfies an orientation threshold. The method may comprise terminating or reducing a supply of at least one of electrical stimuli or motorized assistance to at least the portion of the paretic leg of the user. For example, terminating or reducing the supply may be based on the orientation satisfying the orientation threshold and the angular velocity satisfying the angular velocity threshold.

In certain examples, a method for terminating or reducing assistance to a paretic leg may be provided. The method may comprise receiving kinematic data for a portion of a paretic leg of a user. The kinematic data may comprise velocity data for the portion of the paretic leg and a position for the portion of the paretic leg. The method may comprise determining the angular velocity data satisfies an angular velocity threshold. The method may comprise terminating or reducing the supply of at least one of electrical stimuli or motorized assistance to at least the portion of the paretic leg of the user. For example, terminating or reducing the supply may be based on the position data of the portion of the paretic leg and the angular velocity data satisfying the angular velocity threshold.

In certain examples, a method for terminating or reducing assistance to a paretic leg may be provided. The method may comprise receiving first kinematic data for a portion of a paretic leg of a user. For example, the first kinematic data may comprise a first orientation angle for the first portion of the paretic leg. The method may comprise receiving second kinematic data for a second portion of the paretic leg of the user. For example, the second kinematic data may comprise a second orientation angle for the second portion of the paretic leg. The method may comprise determining a difference between the second orientation angle and the first orientation angle satisfies a threshold. The method may comprise terminating or reducing the supply of at least one of electrical stimuli or motorized assistance to at least the first portion of the paretic leg of the user. For example, terminating or reducing the supply may be based on the difference between the second orientation angle and the first orientation angle satisfying the threshold.

In certain examples, a method for terminating or reducing assistance to a paretic leg may be provided. The method may comprise receiving first kinematic data for a first portion of a paretic leg of a user. For example, the first kinematic data may comprise first orientation data for the first portion of the paretic leg. The method may comprise receiving second kinematic data for a second portion of the paretic leg of the user. For example, the second kinematic data comprises second orientation data for the second portion of the paretic leg. The method may comprise determining, a limb orientation value. For example, the limb orientation value may be based on the first orientation data and the second orientation data. The method may comprise determining the limb orientation value satisfies a threshold. The method may comprise terminating or reducing a supply of at least one of electrical stimuli or motorized assistance to at least the portion of the paretic leg. For example, terminating or reducing the supply may be based on the limb orientation value satisfying the threshold.

This summary is not intended to identify critical or essential features of the disclosure, but merely to summarize certain features and variations thereof. Other details and features will be described in the sections that follow. Additional advantages of the disclosure will be set forth in part in the description that follows, and in part will be obvious from the description, or may be learned by practice of the concepts described in this disclosure. The advantages of the concepts described in this disclosure will be realized and attained by means of the elements and combinations particularly pointed out in the appended claims. It is to be understood that both the foregoing general description and the following detailed description are exemplary and explanatory only and do not restrict the scope of the claims.

### DESCRIPTION OF THE DRAWINGS

The accompanying drawings, which are incorporated in and constitute a part of the present description serve to explain the principles of the methods, apparatuses, and systems described herein:

FIG. 1 shows an example system for providing or terminating assistance for leg movement;

FIG. 2 shows an example system for providing or terminating assistance for leg movement;

FIG. 3 shows an example system for providing or terminating assistance for prosthetic leg movement;

FIG. 4 shows an example method for providing assistance for leg movement;

FIG. 5 shows an example method for providing assistance for leg movement;

FIG. 6 shows an example method for terminating or reducing assistance for leg movement;

FIG. 7 shows an example method for terminating or reducing assistance for leg movement;

FIG. 8 shows an example method for terminating or reducing assistance for leg movement;

FIG. 9 shows an example method for terminating or reducing assistance for leg movement;

FIG. 10 shows an example method for terminating or reducing assistance for leg movement;

FIG. 11 shows an example method for terminating or reducing assistance for leg movement; and FIG. 12 show example system for providing or terminating assistance for leg movement.

### DETAILED DESCRIPTION

Before the present methods, systems, and apparatuses are disclosed and described, it is to be understood that the methods, systems, and apparatuses are not limited to specific methods, specific components, or to particular implementations. It is also to be understood that the terminology used herein is for the purpose of describing particular embodiments only, and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. Furthermore, descriptions of an event or circumstance without use of “optional” or “optionally” does not mean that the described event does occur, must occur, or is necessary to the operation of the apparatus or system or required for the performance of the method.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods, apparatuses, and systems. This applies to all aspects of this application including, but not limited to, steps in disclosed methods. Thus, if there are a variety of additional steps that may be performed it is understood that each of these additional steps may be performed with any specific embodiment or combination of embodiments of the disclosed methods.

The present methods, systems, and apparatuses may be understood more readily by reference to the following detailed description of example embodiments and the examples included therein and to the Figures and their previous and following description.

As will be appreciated by one skilled in the art, one or more of the methods, systems, and apparatuses described herein may take the form of an entirely hardware embodiment, an entirely software embodiment, or an embodiment combining software and hardware aspects. Furthermore, the methods, systems, and apparatuses may take the form of a computer program product on a computer-readable storage medium (e.g., a non-transitory computer-readable medium) and having computer-readable program instructions (e.g., computer software) embodied in the storage medium. Any suitable computer-readable storage medium (e.g., a non-transitory computer-readable medium) may be utilized including hard disks, CD-ROMs, optical storage devices, flash drive, SD card or similar non-volatile memory card, or magnetic storage devices.

Embodiments of the methods, systems, and apparatuses are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, may be implemented by computer program instructions. These computer program instructions may be loaded onto a microcontroller, general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce functions on an article of manufacture including computer-readable instructions for implementing the function specified in the flowchart block or blocks. The computer program instructions may also be loaded onto a microcontroller, computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, may be implemented by special purpose hardware-based computer systems or one or more microcontrollers that perform the specified functions or steps, or combinations of special purpose hardware and computer instructions.

Hereinafter, various embodiments of the present disclosure will be described with reference to the accompanying drawings. As used herein, the term “user” may indicate a person or patient.

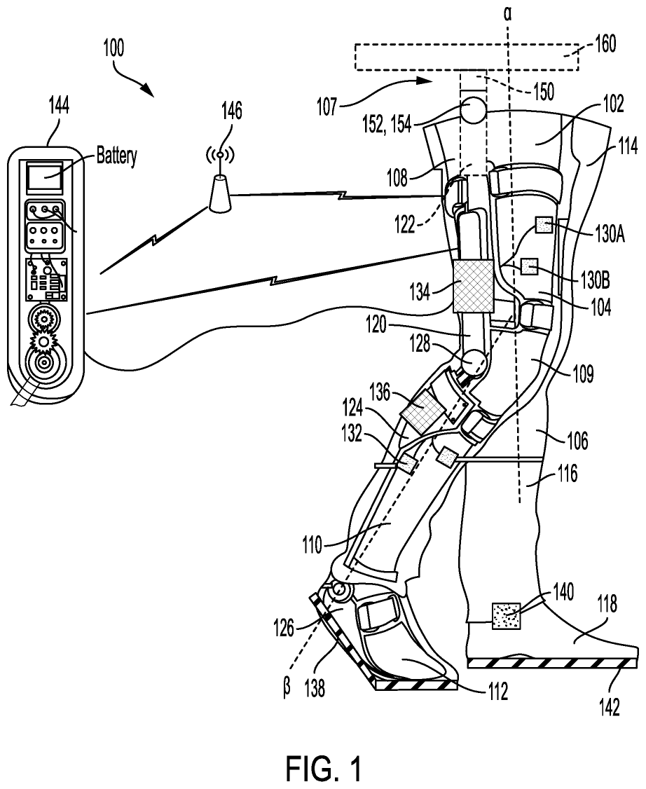

FIG. 1 shows an example system 100 for providing or terminating assistance for leg movement. For example, the assistance may be provided to a user 102, such as a person. The user 102 may have a paretic leg 104 (e.g., a leg suffering partial paralysis) and a non-paretic leg 106. While the example of FIG. 1 shows the right leg of the user 102 being the paretic leg 104, this is for example purposes only. The paretic leg 104 may have been caused by a stroke or other ailment or injury suffered by the user 102.

The paretic leg 104 may comprise a pelvic (or hip) portion 107, a thigh portion 108, a knee portion 109, a shank portion 110, and a foot 112. For example, the thigh portion 108 may be the portion of the paretic leg 104 between the hip or pelvic portion 107 and the knee portion 109 of the paretic leg 104. For example, the shank portion 110 may be the portion of the paretic leg 104 between the knee portion 109 and the foot 112 of the paretic leg 104. The knee portion 109 may be the portion of the paretic leg 104 providing an axis of rotation for the shank portion 110 with respect to the thigh portion 108. A hip may be the portion of the paretic leg 104 providing an axis of rotation for the thigh portion 108 with respect to the pelvic portion 107.

The non-paretic leg 106 may comprise a thigh portion 114, a shank portion 116, and a foot 118. For example, the thigh portion 114 may be the portion of the non-paretic leg 106 between the hip and the knee of the non-paretic leg 106. For example, the shank portion 116 may be the portion of the non-paretic leg 106 between the knee and the foot 118 of the non-paretic leg 106.

The system 100 may comprise a brace 120. For example, the brace 120 may be a leg brace. For example, the brace 120 may be configured to be attached to the paretic leg 104 of the user 102. The brace 120 may be made of one or more of plastic or metal components. For example, the brace 120 may comprise one or more straps, belts, or the like for removably attaching the brace 120 to the paretic leg 104 or another portion (e.g., the waist) of the user 102. For example, the one or more straps may comprise hook and loop straps that provide adjustability in attaching the brace 120 to the paretic leg 104 or another portion of the user 102.

The brace 120 may comprise one or more sections. For example, the brace 120 may comprise a thigh section 122, a shank section 124, and a foot support section 126. In certain examples, the brace 120 may also comprise a hip section 150, and a waist section 160 for attaching the brace 120 around the user's waist. The thigh section 122 may be movably coupled to the shank section 124 and may be configured to move or rotate with respect to the shank section 124. In certain examples, the thigh section 122 may also be movably coupled to the hip section 150 and may be configured to move or rotate with respect to the hip section 150. The thigh section 122 may include an elongated support member. The elongated support member may be configured to extend along at least a portion of the thigh portion (e.g., upper leg) 108 of the user 102. For example, the thigh section 122 may be configured to be positioned along an outer side of the thigh portion 108 of the paretic leg 104. The thigh section 122 may comprise one or more straps, belts, or the like for removably attaching the thigh section 122 to the thigh portion 108 of the paretic leg 104. For example, the one or more straps may comprise hook and loop straps that provide adjustability in attaching the thigh section 122 to the thigh portion 108.

The shank section 124 may be movably coupled to the thigh section 122 and may be configured to move or rotate with respect to the thigh section 122. The shank section 124 may be movably coupled to the foot support section 126 and may be configured to move or rotate with respect to the foot support section 126. The shank section 122 may include an elongated support member. The elongated support member may be configured to extend along at least a portion of the shank portion (e.g., lower leg) 110 of the paretic 104. For example, the shank section 124 may be configured to be positioned along an outer side and/or back side of the shank portion 110 of the paretic leg 104. The shank section 124 may comprise one or more straps, belts, or the like for removably attaching the shank section 124 to the shank portion 110 of the paretic leg 104. For example, the one or more straps may comprise hook and loop straps that provide adjustability in attaching the shank section 124 to the shank portion 110.

The foot support section 126 may be movably coupled to the shank section 124 and may be configured to move or rotate with respect to the shank section 124. The foot support section 126 may include one or more panels. The one or more panels may comprise a bottom panel configured to contact a bottom side of the foot 112 of the paretic leg 104. The one or more panels may also comprise one or more side panels or a rear panel extending up from the bottom panel. The foot support section 126 may comprise one or more straps, belts, or the like for removably attaching the foot support section 126 to the foot 112 of the user 102. For example, the one or more straps may comprise hook and loop straps that provide adjustability in attaching the foot support section 126 to the foot 112.

The hip section 150 may be movably coupled to the thigh section 122 and may be configured to move or rotate with respect to the thigh section 122. The hip section 150 may extend from the thigh section 122 to the waist section 160. The hip section 150 may include a support member (e.g., an elongated support member). The support member may be configured to extend along at least a portion of the pelvic portion 107 of the paretic leg 104. For example, the hip section 150 may be configured to be positioned along an outer side of the pelvic portion 107 of the paretic leg 104.

The waist section 160 may be coupled to the hip section 150. The waist section 160 may be configured to extend around the waist or trunk/torso of the user 102. The waist section 160 may comprise one or more straps, belts, or the like for removably attaching the waist section 160 around the waist/torso/trunk of the user 102. For example, the one or more straps may comprise hook and loop straps that provide adjustability in attaching the waist section 160 to the waist/torso/trunk of the user 102.

The brace 120 may comprise one or more motors 128. The one or more motors 128 may be positioned at or near an axis of rotation between the thigh section 122 and the shank section 124. In certain examples, another one or more motors 152 may be positioned at or near an axis of rotation between the thigh section 122 and the hip section 150. For example, the one or more motors 152 may be provided for users 102 that have limited active hip motion. The one or more motors 128 may be configured to provide motorized assistance with respect to the shank portion 110 rotating with respect to the thigh portion 108 of the paretic leg 104 by providing motorized assistance for the shank section 124 to rotate with respect to the thigh section 122 of the brace 120. In other examples, the one or more motors 128 may be configured to provide motorized resistance with respect to the shank portion 110 rotating with respect to the thigh portion 108 by providing motorized resistance against the shank section 124 rotating with respect to the thigh section 122 of the brace 120. The one or more motors 152 may be configured to provide motorized assistance with respect to the thigh portion 108 rotating with respect to the pelvic portion 107 of the paretic leg 104 by providing motorized assistance for the thigh section 122 to rotate with respect to the hip section 150 of the brace 120. While one example of providing motorized assistance for the thigh portion 108 with respect to the pelvic portion 107, other examples are possible. For example, cabling could be attached to textiles worn on the leg of the user 102 to generate the torques for mobilizing the thing portion 108 with respect to the pelvic portion 107. For example, the one or more motors 152 may provide motorized assistance with hip flexion at the end of the terminal stance phase and then during early, mid, and terminal swing. Motorized assistance may reduce during terminal swing and the one or more motors 152 may provide motorized assistance with hip/thigh extension from heel strike to midstance. In other examples, the one or more motors 152 may be configured to provide motorized resistance with respect to the thigh portion 108 rotating with respect to the pelvic portion 107 by providing motorized resistance against the thigh section 122 rotating with respect to the hip section 150 of the brace 120.

The one or more motors 128 may include or be operably coupled to a sensor. For example the sensor may be an encoder. The sensor may provide rotational data indicating the amount of rotation of the shank section 124 with respect to the thigh section 122. The one or more motors 128 may be electrically coupled to a power source (not shown). The one or more motors 152 may include or be operably coupled to a sensor 154. For example, the sensor 154 may be an encoder. The sensor 154 may provide rotational data indicating the amount of rotation of the thigh portion 108 with respect to the pelvic portion 107 of the paretic leg 104. The one or more motors 152 and the sensor 154 may be electrically coupled to a power source (not shown). The power source may be coupled to the brace 120 and may be configured to provide electrical power to one or more components of the brace 120. For example, the power source may be a battery or battery pack, such as a rechargeable battery. For example, the power source may be one or more of a lead-acid rechargeable battery, a nickel-cadmium rechargeable battery, a nickel-metal hydride rechargeable battery, or a lithium-ion rechargeable battery.

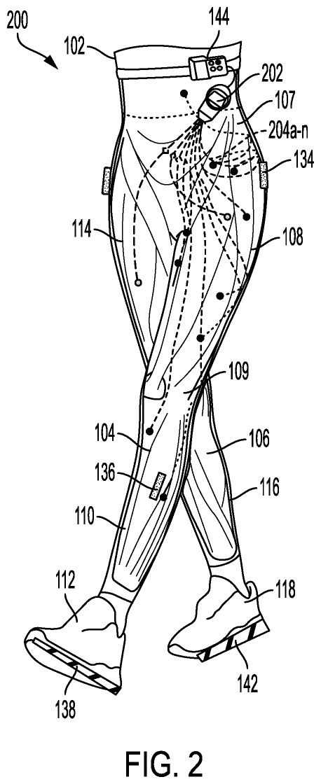

The brace 120 may comprise one or more electrodes 130A-B, 132. The one or more electrodes may be electrically coupled to a pulse generator (not shown but similar to the pulse generator of FIG. 2) that provides an electrical pulse to the electrodes 130A-B, 132. The one or more electrodes 130A-B may be positioned at one or more locations along the thigh portion 108 of the paretic leg 104. The one or more electrodes 132 may be positioned at one or more locations along the shank portion 110 of the paretic leg 104. The one or more electrodes 130A-B, 132 may be configured to provide electrical stimuli to the muscles of the thigh portion 108 and/or shank portion 110 and/or any other portion or portions of the paretic leg 104 in order to provide assistance with rotation and/or movement of the thigh portion 108 and/or shank portion 110 of the leg 104. The one or more electrodes 130A-B, 132 may be operably coupled to the sensor for the motor 128 and/or the sensor 154. The sensor for the motor 128 may provide rotational data indicating the amount of rotation of the shank section 124 with respect to the thigh section 122. The sensor 154 may provide rotational data indicating the amount of rotation of the thigh section 122 with respect to the hip section 150. The one or more electrodes 130A-B, 132 may be electrically coupled to the power source. Additional electrodes (not shown) may be provided to any of the portions of the paretic leg 104 for providing electrical stimuli to those portions of the paretic leg 104.

The brace 120 may comprise a thigh sensor 134. The thigh sensor 134 may be positioned along a portion of the thigh section 122 of the brace 120. For example, the thigh sensor 134 may be coupled to the elongated member of the thigh section 122. For example, the thigh sensor 134 may be an inertial measurement unit or another form of sensor. For example, the thigh sensor 134 may comprise multiple sensors for detecting certain data related to the thigh portion 108 of the paretic leg 104. For example, the thigh sensor may generate or collect data related to the thigh portion 108, the data comprising one or more of acceleration data indicating an acceleration for the thigh portion 108, angular velocity data indicating an angular velocity for the thigh portion 108, orientation data indicating an orientation of the thigh portion 108, and/or position data indicating a position of the thigh portion 108. For example, the thigh sensor 134 may collect data related to the muscle activity along the thigh portion 108 of the paretic leg 104. The muscle activity data may indicate a muscle activity level for the thigh portion 108. The muscle activity level may be compared to a muscle activity threshold. If the muscle activity level satisfies (e.g., is greater than or greater than or equal to) the muscle activity threshold the muscle activity level may indicate an initiation of a phase of the gate cycle and/or a transition from one phase to another phase of the gait cycle.

For example, the orientation data may indicate the an angle of the thigh portion 108 as taken along an elongated axis (α) of the thigh portion 108 as compared to a vertical axis or a horizontal axis. The thigh sensor 134 may be electrically coupled to the power source. The thigh sensor 134 may be communicably coupled to the control computing device 144 and may send the one or more of the acceleration data, velocity data (e.g., angular velocity data), orientation data, muscle activity data, and position data to the control computing device 144.

The brace 120 may comprise a shank sensor 136. The shank sensor 136 may be positioned along a portion of the shank section 124 of the brace 120. For example, the shank sensor 136 may be coupled to the elongated member of the shank section 124. For example, the shank sensor 136 may be an inertial measurement unit or another form of sensor. For example, the shank sensor 136 may comprise multiple sensors for detecting certain data related to the shank portion 110 of the paretic leg 104. For example, the shank sensor 136 may generate or collect data related to the shank portion 110, the data comprising one or more of acceleration data indicating an acceleration for the shank portion 110, velocity data indicating an angular velocity for the shank portion 110, orientation data indicating an orientation of the shank portion 110, and/or position data indicating a position of the shank portion 110. For example, the shank sensor 136 may collect data related to the muscle activity along the shank portion 110 of the paretic leg 104. The muscle activity data may indicate a muscle activity level for the shank portion 108. The muscle activity level may be compared to a second muscle activity threshold. If the muscle activity level satisfies (e.g., is greater than or greater than or equal to) the second muscle activity threshold the muscle activity level may indicate an initiation of a phase of the gate cycle and/or a transition from one phase to another phase of the gait cycle.

For example, the orientation data may indicate the an angle of the shank portion 110 as taken along an elongated axis (β) of the shank portion 110 as compared to a vertical axis or a horizontal axis. The shank sensor 136 may be electrically coupled to the power source. The shank sensor 136 may be communicably coupled to the control computing device 144 and may send the one or more of the acceleration data, velocity data, orientation data, muscle activity data, and/or position data to the control computing device 144.

The brace 120 may comprise a heel-strike sensor 138. The heel-strike sensor 138 may be positioned along a portion of the foot support section 126 of the brace 120. For example, the heel-strike sensor 138 may be coupled to the bottom end or bottom surface of the foot support section 126 or placed on another portion of the paretic leg 104. For example, the heel-strike sensor 138 may be an inertial measurement unit, a contact sensor, a pressure sensor, or another form of sensor. For example, the heel-strike sensor 138 may indicate when the foot support section 126, the heel of the foot 112 or another portion of the foot 112 of the paretic leg 104 contacts a floor surface. The heel-strike sensor 138 may be electrically coupled to the power source. The heel-strike sensor 138 may be communicably coupled to the control computing device 144 and may send the data indicating the contact with the floor surface to the control computing device 144.

The brace 120 may comprise a foot sensor 140. The foot sensor 140 may be positioned along a portion of the foot 118, ankle, shank section 116, or another portion of the non-paretic leg 106. For example, the foot sensor 140 may be an inertial measurement unit or another form of sensor. For example, the foot sensor 140 may comprise multiple sensors for detecting certain data related to the non-paretic leg 106. For example, the foot sensor 140 may generate or collect data related to the foot 118 or shank portion 116, the data comprising one or more of acceleration data indicating an acceleration for the foot 118 or shank portion 116, velocity data indicating an angular velocity for the foot 118 or shank portion 116, orientation data indicating an orientation of the foot 118 or shank portion 116, heel-strike or contact information for the foot 118 along the floor surface and/or position data indicating a position of the foot 118 or shank portion 116. The foot sensor 140 may be electrically coupled to the power source. The foot sensor 140 may be communicably coupled to the control computing device 144 and may send the one or more of the acceleration data, angular velocity data, orientation data, heel-strike contact data, muscle activity data, and/or position data to the control computing device 144.

The brace 120 may comprise a hip or pelvic (“hip”) sensor. The hip sensor may be positioned along the hip or pelvic region of the user 102. For example, the hip sensor may comprise multiple sensors for detecting certain data related to the hip or pelvic region of the paretic leg 104. For example, the hip sensor may generate or collect data related to the hip or pelvic region, the data comprising one or more of acceleration data indicating an acceleration for the hip region, velocity data (e.g., angular velocity data) indicating a velocity or angular velocity for the hip region, orientation data indicating an orientation of the hip region, and/or position data indicating a position of the hip region. For example, the hip sensor may collect data related to the muscle activity along the hip or pelvic region of the paretic leg 104. The muscle activity data may indicate a muscle activity level for the hip or pelvic region. The muscle activity level may be compared to a third muscle activity threshold. If the muscle activity level satisfies (e.g., is greater than or greater than or equal to) the third muscle activity threshold the muscle activity level may indicate an initiation of a phase of the gate cycle and/or a transition from one phase to another phase of the gait cycle.

The system 100 may comprise a heel-strike sensor 142. The heel-strike sensor 142 may be positioned along a portion of a shoe or foot covering of the foot 118 of the non-paretic leg 106. For example, the heel-strike sensor 142 may be coupled to the bottom end or bottom surface of a shoe. For example, the heel-strike sensor 142 may be an inertial measurement unit, a contact sensor, a pressure sensor, or another form of sensor. For example, the heel-strike sensor 142 may indicate when the heel of the foot 118 or another portion of the foot 118 or shoe of the non-paretic leg 106 contacts the floor surface. The heel-strike sensor 142 may be electrically coupled to the power source. The heel-strike sensor 142 may be communicably coupled to the control computing device 144 and may send the data indicating the contact with the floor surface to the control computing device 144.

The system 100 may comprise a control computing device 144. The control computing device 144 may be a form of computer. The control computing device 144 may be communicably coupled to the sensors 134-142, 154, the motors 128, 152, and/or the electrodes 130A-B, 132. The control computing device 144 may communicate with the sensors 134-142, 154, the motors 128, 152, and/or the electrodes 130A-B, 132 via wired or wireless communication. For example, the control computing device 144 may communicate wirelessly via one or more of WI-FI, Bluetooth, Bluetooth Low Energy (BLE), Zigbee, or any other known wireless protocol with the sensors 134-142, 154, the motors 128, 152, and the electrodes 130A-B, 132. For example, the control computing device 144 may communicate wirelessly with the brace 120 either directly (e.g., via Bluetooth, BLE, Zigbee, etc.) or via a network (e.g., a WI-FI network), such as via the network device 146 or another network. For example, the control computing device 144 may be a user device, such as a desktop computer, a laptop computer, a smart device, a mobile device (e.g., a mobile phone (e.g., a smart phone), a tablet device, a smart watch, etc.), and/or the like.

The control computing device 144 may comprise one or more processors, one or more memory modules, a power source, a communications module, and/or one or more selection buttons or switches. For example, the one or more processors may comprise any one or more of microcontrollers, microprocessors, or embedded processors. The one or more processers may be configured to receive the data from the one or more sensors 134-142 and determine whether to initiate, terminate, reduce, and/or continue providing one or more of electrical stimuli or motorized assistance at the brace 120. For example, the power source may be a battery, such as a rechargeable battery. For example, the communications module may comprise a transmitter, receiver, or transceiver. The communications module may be configured to receive data from one or more of the sensors 134-142, 154. The communications module may be further configured to send instructions to the motors 128, 152 and/or the electrodes 130A-B, 132 to provide motorized assistance and/or electrical stimuli to the paretic leg 104.

The system 100 may comprise the network device 146. The network device 146 may comprise a local gateway (e.g., router, modem, switch, hub, combinations thereof, and the like) configured to connect (or facilitate a connection (e.g., a communication session) between) a local area network (e.g., a LAN) to a wide area network (e.g., a WAN). The network device 146 may configured to receive incoming data (e.g., data packets or other signals) from the brace 120 (e.g., one or more of the sensors 134-142) and route the data to the control computing device 144 and may be configured to receive incoming data from the control computing device 144 and route that data to the brace 120 (e.g., one or more of the motors 128, 152 and/or electrodes 130A-B, 132). The network device 146 may be configured to communicate with a network. The network device 146 may be configured for communication with the network via a variety of protocols, such as IP, transmission control protocol, file transfer protocol, session initiation protocol, voice-over IP (e.g., VoIP), combinations thereof, and the like. The network device 146 may be configured to facilitate network access via a variety of communication protocols and standards.

FIG. 2 shows an example system 200 for providing or terminating assistance for leg movement. For example, the assistance may be provided to the user 102. While some elements may not be specifically shown, the system 200 of FIG. 2 may comprise the paretic leg 104, non-paretic leg 106, sensors 134-142, and control computing device 144 as described in FIG. 1. The system 200 may further comprise a pulse generator 202. The pulse generator 202 may be configured to generate electrical pulses (stimuli) for one or more electrodes 204a-n. The pulse generator 202 may be implanted in the user 102. For example, the pulse generator 202 may be implanted in the thigh portion 108 of the paretic leg 106. In other examples, the pulse generator 202 may be implanted in another portion of the body of the user 102. For example, the pulse generator 202 may be communicably coupled to the control computing device 144 via wired or wireless communication. For example, the pulse generator 202 may be electrically coupled to the one or more electrodes 204a-n. Each of the one or more electrodes 204a-n may be implanted within a portion of the legs (e.g., the paretic leg 104 and/or the non-paretic leg 106) of the user 102 to provide electrical stimuli to the muscles of the user 102 and/or monitor the activity of the user 102.

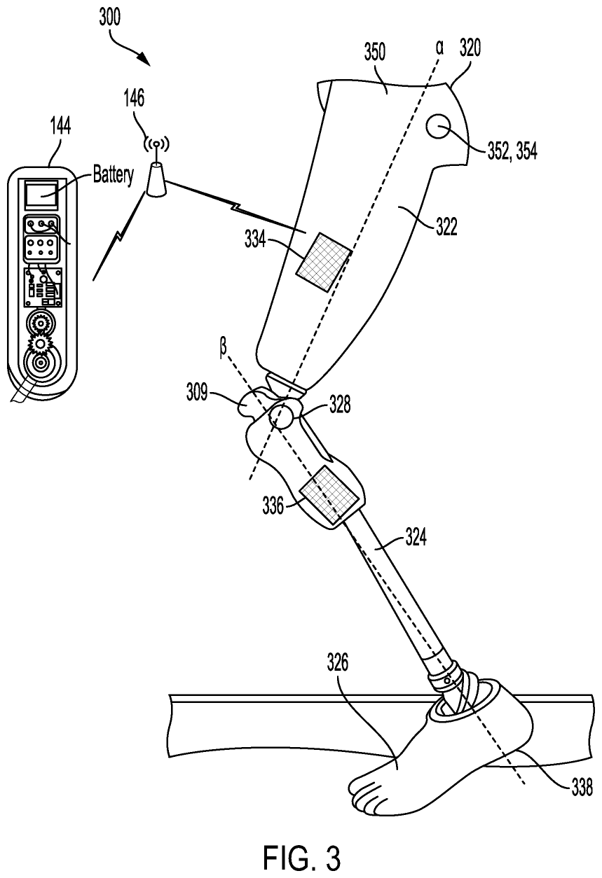

FIG. 3 shows an example system 300 for providing or terminating assistance for movement of a prosthetic leg. For example, the assistance may be provided to a user, such as a person. For example, the user may be substantially similar to the user 102 except that the user may have had all or a portion of their leg amputated. For example, the user may further have another leg, substantially the same as the non-paretic leg of 106 of FIG. 1 and the system 300 may include the same sensors as described herein with regard to the non-paretic leg 106 on the other leg of the user.

The amputated leg may comprise a pelvic (or hip) portion and, in certain examples, a partial thigh portion. The other leg of the user may comprise a thigh portion, a shank portion, and a foot. For example, the thigh portion may be the portion of the other leg between the hip and the knee of the other leg. For example, the shank portion may be the portion of the other leg between the knee and the foot of the other leg of the user.

The system 300 may comprise a prosthetic leg 320. For example, the prosthetic leg 320 may be configured to be attached to the remaining portion of the amputated leg of the user. The prosthetic leg 320 may be made of one or more of plastic or metal components. For example, the prosthetic leg 320 may comprise one or more straps, belts, or the like for removably attaching the prosthetic leg 320 to the remaining portion of the amputated leg of the user. For example, the one or more straps may comprise hook and loop straps that provide adjustability in attaching the prosthetic leg 320 to the remaining portion of the leg of the user.

The prosthetic leg 320 may comprise one or more sections. For example, the prosthetic leg 320 may comprise a thigh section 322, a shank section 324, and a foot section 326. In certain examples, the prosthetic leg 320 may also comprise a hip section 350. The thigh section 322 may be movably coupled to the shank section 324 at a knee joint 309 and may be configured to move or rotate with respect to the shank section 324. In certain examples, the thigh section 322 may also be movably coupled to the hip section 350 and may be configured to move or rotate with respect to the hip section 350. The thigh section 322 may include an elongated cavity for receiving the remaining portion of the amputated leg. The thigh section 322 may comprise one or more straps, belts, or the like for removably attaching the thigh section 322 to the remaining portion of the amputated leg. For example, the one or more straps may comprise hook and loop straps that provide adjustability in attaching the thigh section 322 to the remaining portion of the amputated leg.

The shank section 324 may be movably coupled to the thigh section 322 and may be configured to move or rotate with respect to the thigh section 322 about the knee joint 309. The shank section 324 may be movably coupled to the foot section 326 and may be configured to move or rotate with respect to the foot section 326. The shank section 324 may include an elongated support member.

The foot section 326 may be movably coupled to the shank section 324 and may be configured to move or rotate with respect to the shank section 324. The foot section 326 may include one or more panels.

The hip section 350, if optionally included, may be movably coupled to the thigh section 322 and may be configured to move or rotate with respect to the thigh section 322. The hip section 350 may extend above and/or horizontally out from the thigh section 322. The hip section 350 may include a support member (e.g., an elongated support member). The support member may be configured to extend along at least a portion of the pelvic portion of the remaining portion of the amputated leg of the user.

The prosthetic leg 320 may comprise one or more motors 328. The one or more motors 328 may be positioned at or near an axis of rotation between the thigh section 322 and the shank section 324, such as along the knee portion 309. In certain examples, another one or more motors 352 may be positioned at or near an axis of rotation between the thigh section 322 and the hip section 350. For example, the one or more motors 352 may be provided for users that have limited active hip or thigh motion. The one or more motors 328 may be configured to provide motorized assistance for the shank section 324 to rotate with respect to the thigh section 322 of the prosthetic leg 320. The one or more motors 352 may be configured to provide motorized assistance for the thigh section 322 to rotate with respect to the hip section 350 of the brace prosthetic leg 320. For example, the one or more motors 352 may provide motorized assistance with hip flexion at the end of the terminal stance phase and then during early, mid, and terminal swing. Motorized assistance may reduce during terminal swing and the one or more motors 352 may provide motorized assistance with hip/thigh extension from heel strike to midstance. In other examples, the one or more motors 352 may be configured to provide motorized resistance with respect to the remaining portion of the thigh portion of the user rotating with respect to the pelvic portion of the user by providing motorized resistance against the thigh section 322 rotating with respect to the hip section 350 of the prosthetic leg 320.

The one or more motors 328 may include or be operably coupled to a sensor. For example the sensor may be an encoder. The sensor may provide rotational data indicating the amount of rotation of the shank section 324 with respect to the thigh section 322. The one or more motors 328 may be electrically coupled to a power source (not shown). The one or more motors 352 may include or be operably coupled to a sensor 354. For example, the sensor 354 may be an encoder. The sensor 354 may provide rotational data indicating the amount of rotation of the thigh section 322 with respect to the hip section 350 of the prosthetic leg 320. The one or more motors 352 and the sensor 354 may be electrically coupled to a power source (not shown). The power source may be coupled to the prosthetic leg 320 and may be configured to provide electrical power to one or more components of the prosthetic leg 320. For example, the power source may be a battery or battery pack, such as a rechargeable battery. For example, the power source may be one or more of a lead-acid rechargeable battery, a nickel-cadmium rechargeable battery, a nickel-metal hydride rechargeable battery, or a lithium-ion rechargeable battery.

The brace 120 may comprise one or more electrodes. The one or more electrodes may be electrically coupled to a pulse generator (not shown but similar to the pulse generator of FIG. 2) that provides an electrical pulse to the electrodes. The one or more electrodes may be positioned at one or more locations along the remaining portion of the thigh of the amputated leg. The one or more electrodes may be configured to provide electrical stimuli to the muscles of the remainder of the thigh portion of the amputated leg in order to provide assistance with rotation and/or movement of the thigh. The one or more electrodes may be operably coupled to the sensor for the motor 328 and/or the sensor 354. The sensor for the motor 328 may provide rotational data indicating the amount of rotation of the shank section 324 with respect to the thigh section 322. The sensor 354 may provide rotational data indicating the amount of rotation of the thigh section 322 with respect to the hip section 350. The one or more electrodes may be electrically coupled to the power source.

The prosthetic leg 320 may comprise a thigh sensor 334. The thigh sensor 334 may be positioned along a portion of the thigh section 322 of the prosthetic leg 320. For example, the thigh sensor 334 may be coupled to the elongated member of the thigh section 322. For example, the thigh sensor 334 may be an inertial measurement unit or another form of sensor. For example, the thigh sensor 334 may comprise multiple sensors for detecting certain data related to the movement of the thigh section 322. For example, the thigh sensor may generate or collect data related to the thigh section 322, the data comprising one or more of acceleration data indicating an acceleration for the thigh section 322, angular velocity data indicating an angular velocity for the thigh section 322, orientation data indicating an orientation of the thigh section 322, and/or position data indicating a position of the thigh section 322. For example, the thigh sensor 334 may collect data related to the muscle activity along the thigh section 322 of the prosthetic leg 320. The muscle activity data may indicate a muscle activity level for the remaining portion of the thigh of the amputated leg. The muscle activity level may be compared to a muscle activity threshold. If the muscle activity level satisfies (e.g., is greater than or greater than or equal to) the muscle activity threshold the muscle activity level may indicate an initiation of a phase of the gate cycle and/or a transition from one phase to another phase of the gait cycle.

For example, the orientation data may indicate the an angle of the thigh section 322 as taken along an elongated axis (α) of the thigh section 322 as compared to a vertical axis or a horizontal axis. The thigh sensor 334 may be electrically coupled to the power source. The thigh sensor 334 may be communicably coupled to the control computing device 144 and may send the one or more of the acceleration data, velocity data (e.g., angular velocity data), orientation data, muscle activity data, and position data to the control computing device 144.

The prosthetic leg 320 may comprise a shank sensor 336. The shank sensor 336 may be positioned along a portion of the shank section 324 of the prosthetic leg 320. For example, the shank sensor 336 may be coupled to the elongated member of the shank section 324. For example, the shank sensor 336 may be an inertial measurement unit or another form of sensor. For example, the shank sensor 336 may comprise multiple sensors for detecting certain data related to the shank section 324. For example, the shank sensor 336 may generate or collect data related to the shank section 324, the data comprising one or more of acceleration data indicating an acceleration for the shank section 324, velocity data indicating an angular velocity for the shank section 324, orientation data indicating an orientation of the shank section 324, and/or position data indicating a position of the shank section 324.

For example, the orientation data may indicate the an angle of the shank section 324 as taken along an elongated axis (β) of the shank section 324 as compared to a vertical axis or a horizontal axis. The shank sensor 336 may be electrically coupled to the power source. The shank sensor 336 may be communicably coupled to the control computing device 144 and may send the one or more of the acceleration data, velocity data, orientation data, muscle activity data, and/or position data to the control computing device 144.

The prosthetic leg 320 may comprise a heel-strike sensor 338. The heel-strike sensor 338 may be positioned along a portion of the foot section 326 of the prosthetic leg 320. For example, the heel-strike sensor 338 may be coupled to the bottom end or bottom surface of the foot section 326. For example, the heel-strike sensor 338 may be an inertial measurement unit, a contact sensor, a pressure sensor, or another form of sensor. For example, the heel-strike sensor 338 may indicate when the foot section 326, the heel of the foot section 326 or another portion of the foot section 326 contacts a floor surface. The heel-strike sensor 338 may be electrically coupled to the power source. The heel-strike sensor 338 may be communicably coupled to the control computing device 144 and may send the data indicating the contact with the floor surface to the control computing device 144.

The system 300 may comprise a foot sensor substantially the same as the foot sensor 140 of FIG. 1. The foot sensor may be positioned along a portion of the foot, ankle, shank section, or another portion of the other leg of the user. For example, the foot sensor may be an inertial measurement unit or another form of sensor. For example, the foot sensor may comprise multiple sensors for detecting certain data related to the other leg of the user. For example, the foot sensor may generate or collect data related to the foot or shank portion of the other leg of the user, the data comprising one or more of acceleration data indicating an acceleration for the foot or shank portion, velocity data indicating an angular velocity for the foot or shank portion, orientation data indicating an orientation of the foot or shank portion, heel-strike or contact information for the foot along the floor surface and/or position data indicating a position of the foot or shank portion. The foot sensor may be electrically coupled to the power source. The foot sensor may be communicably coupled to the control computing device 144 and may send the one or more of the acceleration data, angular velocity data, orientation data, heel-strike contact data, muscle activity data, and/or position data to the control computing device 144.

The system 300 may comprise a heel-strike sensor substantially the same as the heel-strike sensor 142 of FIG. 1. The heel-strike sensor may be positioned along a portion of a shoe or foot covering of the foot of the other leg of the user. For example, the heel-strike sensor may be coupled to the bottom end or bottom surface of a shoe. For example, the heel-strike sensor may be an inertial measurement unit, a contact sensor, a pressure sensor, or another form of sensor. For example, the heel-strike sensor may indicate when the heel of the foot or another portion of the foot or shoe of the other leg of the user contacts the floor surface. The heel-strike sensor may be electrically coupled to the power source. The heel-strike sensor may be communicably coupled to the control computing device 144 and may send the data indicating the contact with the floor surface to the control computing device 144.

The system 300 may comprise a control computing device 144. The control computing device 144 may be a form of computer. The control computing device 144 may be communicably coupled to the sensors 334-338, 354, the motors 328, 352, and/or the electrodes. The control computing device 144 may communicate with the sensors 334-338, 354, the motors 328, 352, and/or the electrodes via wired or wireless communication. For example, the control computing device 144 may communicate wirelessly via one or more of WI-FI, Bluetooth, Bluetooth Low Energy (BLE), Zigbee, or any other known wireless protocol with the sensors 334-338, 354, the motors 328, 352, and the electrodes. For example, the control computing device 144 may communicate wirelessly with the prosthetic leg 320 either directly (e.g., via Bluetooth, BLE, Zigbee, etc.) or via a network (e.g., a WI-FI network), such as via the network device 146 or another network. For example, the control computing device 144 may be a user device, such as a desktop computer, a laptop computer, a smart device, a mobile device (e.g., a mobile phone (e.g., a smart phone), a tablet device, a smart watch, etc.), and/or the like.

The control computing device 144 may comprise one or more processors, one or more memory modules, a power source, a communications module, and/or one or more selection buttons or switches. For example, the one or more processors may comprise any one or more of microcontrollers, microprocessors, or embedded processors. The one or more processers may be configured to receive the data from the one or more sensors 334-338, 354 and determine whether to initiate, terminate, reduce, and/or continue providing one or more of electrical stimuli or motorized assistance at the prosthetic leg 320. For example, the power source may be a battery, such as a rechargeable battery. For example, the communications module may comprise a transmitter, receiver, or transceiver. The communications module may be configured to receive data from one or more of the sensors 334-338, 354. The communications module may be further configured to send instructions to the motors 328, 352 and/or the electrodes to provide motorized assistance to the prosthetic leg 320 and/or electrical stimuli to the remaining portion of the thigh of the amputated leg.

The system 300 may comprise the network device 146. The network device 146 may comprise a local gateway (e.g., router, modem, switch, hub, combinations thereof, and the like) configured to connect (or facilitate a connection (e.g., a communication session) between) a local area network (e.g., a LAN) to a wide area network (e.g., a WAN). The network device 146 may configured to receive incoming data (e.g., data packets or other signals) from the prosthetic leg 320 (e.g., one or more of the sensors 334-338, 354) and route the data to the control computing device 144 and may be configured to receive incoming data from the control computing device 144 and route that data to the prosthetic leg 320 (e.g., one or more of the motors 328, 352 and/or electrodes). The network device 146 may be configured to communicate with a network. The network device 146 may be configured for communication with the network via a variety of protocols, such as IP, transmission control protocol, file transfer protocol, session initiation protocol, voice-over IP (e.g., VoIP), combinations thereof, and the like. The network device 146 may be configured to facilitate network access via a variety of communication protocols and standards.

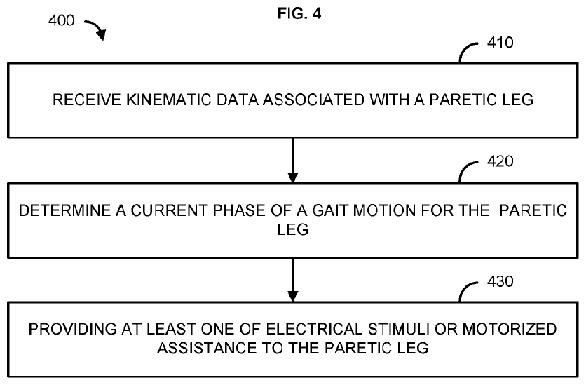

FIG. 4 shows an example method 400 for providing assistance for leg movement. Referring to FIGS. 1-4, the method 400 may be completed by one or more of the control computing device 144, the brace 120 (or prosthetic leg 320), and/or the pulse generator 202. At 410, kinematic data may be received. For example, the kinematic data may be associated with the paretic leg 104 (or the prosthetic leg 320). For example, the kinematic data may have been received from one or more sensors (e.g., the sensors 134-138 or 334-338) receiving data about the paretic leg 104 (or the prosthetic leg 320). For example, the kinematic data may be received by the control computing device 144 from one or more of the sensors 134-138 or 334-338. For example, the kinematic data may comprise one or more of velocity data (e.g., angular velocity data) of all or a portion of the paretic leg 104 (or the prosthetic leg 320), acceleration data of all or a portion of the paretic leg 104 (or the prosthetic leg 320), orientation data of all or a portion of the paretic leg 104 (or the prosthetic leg 320), muscle activity data for all or a portion of the paretic leg 104 (or the remaining portion of the thigh of the amputated leg), or position data for all or a portion of the paretic leg 104 (or the prosthetic leg 320). For example, the kinematic data may comprise a calculated estimated center of mass of the body of the user 102. For example, the portion of the paretic leg 104 (or the prosthetic leg 320) may comprise one of the thigh portion 108 of the paretic leg 104 (or thigh section 322 of the prosthetic leg 320), the shank portion 110 of the paretic leg 104 (or shank section 324 of the prosthetic leg 320), or the foot 112 of the paretic leg (or foot section 326 of the prosthetic leg 320). For example, the kinematic data may comprise an orientation (e.g., an angle of orientation) for the thigh portion 108 of the paretic leg 104 (or thigh section 322 of the prosthetic leg 320). For example, the angle of orientation for the thigh portion 108 (or thigh section 322) may be determined based on the angle between the longitudinal axis α of the thigh portion 108 (or the thigh section 322) and one of a vertical axis and a horizontal axis. For example, the kinematic data may comprise heel-strike or pressure data from the sensor that detects heel-strike 138 or 338.

At 420, a current phase of the gait motion for the paretic leg 104 (or the prosthetic leg 320) may be determined. The current phase of the gait motion may be determined by the control computing device 144 or any other computing device. For example, the current phase of the gait motion may be determined based on the received kinematic data for the paretic leg 104 (or the prosthetic leg 320) and/or the calculated estimate of the center of mass of the body of the user 102. For example, the current phase of the gait motion of the paretic leg 104 (or the prosthetic leg 320) may be determined based on orientation of the thigh portion 108 of the paretic leg 104 (or thigh section 322 of the prosthetic leg 320). For example, the current phase of the gait motion may be determined based on the orientation of the thigh portion 108 (or the thigh section 322) and the orientation of the shank portion 110 of the paretic leg 104 (or the shank section 324 of the prosthetic leg 320). For example, the current phase of the gait motion may be determined based on the heel-strike or pressure data from the heel-strike sensor 138 or 338. For example, the current phase of the gait motion may be determined based on the heel-strike or pressure data from the heel strike sensor 138 or 338, the orientation, muscle activity, or position data for the thigh portion 108 (or the thigh section 322) from the thigh sensor 134 or 334, and/or the orientation, muscle activity, or position data for the shank portion 110 (or shank section 324) from the shank sensor 136 or 336. For example, the current phase of the gait motion of the paretic leg 104 (or the prosthetic leg 320) may be further determined based on the velocity data (e.g., angular velocity data) for the thigh portion 108 (or the thigh section 322) and/or the velocity data (e.g., angular velocity data) for the shank portion 110 of the paretic leg 104 (or the shank section 324 of the prosthetic leg 320). For example, the current phase of the gait motion of the paretic leg 104 (or the prosthetic leg 320) may further be determined based on the calculated estimate of the center of mass of the body of the user 102. For example, the current phase of the gait motion for the paretic leg 104 (or the prosthetic leg 320) may be one of heel-strike, foot flat, midstance, heel off, toe off, swing phase (e.g., initial swing, mid-swing, or terminal swing) or stance.

At 430, at least one of electrical stimuli or motorized assistance may be provided to the paretic leg 104 (or the prosthetic leg 320). For example, the control computing device 144 or another computing device may cause the electrical stimuli and/or the motorized assistance to be provided to the paretic leg 104 (or the prosthetic leg 320). For example, the control computing device 144 may send a signal to one or more of the motors 128, 152 or 328, 352 to provide motorized assistance and/or to the electrodes 130A-B, 132 or the pulse generator 202 to provide electrical stimuli to the paretic leg 104 (or the prosthetic leg 320). The electrical stimuli and/or motorized assistance may be provided based on the current phase of the gait motion of the paretic leg 104 (or the prosthetic leg 320).

For example, the motor 128 or 328 may provide motorized assistance by causing the shank section 124 of the brace 120 (or shank section 324 of the prosthetic leg 320) to rotate with respect to the thigh section 122 of the brace 120 (or thigh section 322 of the prosthetic leg 320). For example, the motor 152 or 352 may provide motorized assistance by causing the thigh section 122 of the brace 120 (or thigh section 322 of the prosthetic leg 320) to rotate with respect to the hip section 150 of the brace 120 (or hip section 350 of the prosthetic leg 320). For example, the electrodes 130A-B, 132 or the pulse generator 202 may provide electrical stimuli to portions of the paretic leg 104 (or the remaining portion of thigh of the amputated leg) by sending electrical pulses into the muscles of the paretic leg 104 (or the remaining portions of thigh of the amputated leg).

For example, during the swing phase, and more particularly during the initial swing to midswing phase of the swing phase, electrical stimulation may be provided to the short head of biceps femoris (hamstring knee flexor) and dorsiflexors and the motor 128 or 328 can provide assistance to flex the knee 109 or knee section 309. For example, during the terminal swing phase of the swing phase, electrical stimulation may be applied to quadriceps and dorsiflexors and motorized assistance can be provided by the motor to help extend the knee 109 (or knee section 309) of the paretic leg 104 (or the prosthetic leg 320).

For example during the stance phase, and more particularly during the loading response and early stance of the stance phase, electrical stimuli provided to the dorsiflexor may be reduced and electrical stimuli provided to the quadriceps may be reduced. Electrical stimuli may be provided to the hamstrings to promote hip extension for the paretic leg 104 (or the remaining portion of thigh of the amputated leg) and the motor 128 or 328 may provide motorized assistance to maintain knee extension.

For example, during the terminal stance of the stance phase, electrical stimuli may be provided to the hamstrings for hip extension and gastrocnemius/soleus for plantarflexion (and knee flexion). The motor 128 or 328 may provide motorized assistance to allow the shift from knee extension to flexion for the paretic leg 104 (or the prosthetic leg 320).

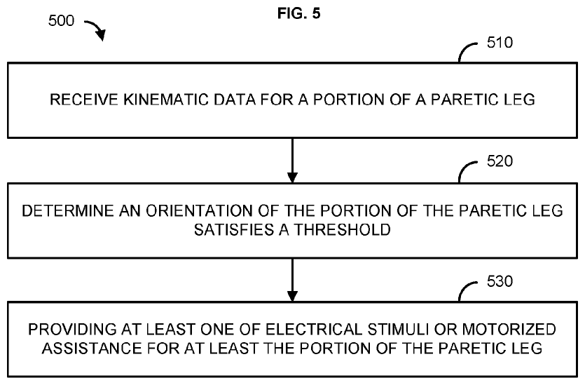

FIG. 5 shows an example method 500 for providing assistance for leg movement. Referring to FIGS. 1-3 and 5, the method 500 may be completed by one or more of the control computing device 144, the brace 120 (or prosthetic leg 320), and/or the pulse generator 202. At 510, kinematic data for a portion of a paretic leg 104 (or the prosthetic leg 320) may be received. For example, the portion of the paretic leg 104 (or the prosthetic leg 320) may be the thigh portion 108 (or the thigh section 322). For example, the kinematic data may have been received from one or more sensors 134-138 or 334-338 (e.g., the thigh sensor 134 or 334) receiving data about the paretic leg 104 (or the prosthetic leg 320). For example, the kinematic data may be received by the control computing device 144 from one or more of the sensors 134-138 or 334-338 (e.g., the thigh sensor 134 or 334).

For example, the kinematic data may comprise orientation data (e.g., an angle of orientation) for the thigh portion 108 of the paretic leg 104 (or thigh section 322 of the prosthetic leg 320). For example, the angle of orientation for the thigh portion 108 (or the thigh section 322) may be determined based on the angle between the longitudinal axis α of the thigh portion 108 (or the thigh section 322) and one of a vertical axis and a horizontal axis. For example, the kinematic data may further comprise acceleration data for the thigh portion 108 of the paretic leg 104 (or the thigh section 322 of the prosthetic leg 320), velocity data (e.g., angular velocity data) for the thigh portion 108 of the paretic leg 104 (or the thigh section 322 of the prosthetic leg 320), muscle activity for the thigh portion 108 of the paretic leg 104 (or the remaining portion of thigh of the amputated leg), and/or position data for the thigh portion 108 of the paretic leg 104 (or the thigh section 322 of the prosthetic leg 320).

The control computing device 144 may determine the orientation (e.g., the angle of orientation) for the thigh portion 108 (or the thigh section 322) based on the kinematic data. For example, the device 144 may parse the angle of orientation from the orientation data of the received kinematic data. For example, the control computing device 144 may further determine the orientation for the thigh portion 108 (or the thigh section 322) based on the calculated estimate of the center of mass of the body of the user 102. At 520, a determination may be made that the angle of orientation for the portion of the paretic leg satisfies an orientation threshold. For example, the determination may be based on the received kinematic data for the paretic leg 104 (or the prosthetic leg 320) and/or the calculated estimate of the center of mass of the body of the user 102. The determination may be made by the control computing device 144 or another computing device. For example, the portion of the paretic leg 104 (or the prosthetic leg 320) may be the thigh portion 108 (or the thigh section 322). In other examples, the portion of the paretic leg 104 (or the prosthetic leg 320) may be the shank portion 110 (or the shank section 324). For example, the control computing device 144 may compare the angle of orientation of the thigh portion 108 (or the thigh section 322) to the orientation threshold to determine if the angle of orientation of the thigh portion 108 (or the thigh section 322) satisfies the orientation threshold. The angle of orientation of the thigh portion 108 (or the thigh section 322) satisfies the threshold if the angle of orientation is one of greater than or greater than or equal to the orientation threshold. In other examples, assistance may be provided based on the angle of orientation not satisfying the threshold and the control computing device 144 may determine that the angle of orientation does not satisfy the orientation threshold based on the angle of orientation being greater than or greater than or equal to the orientation threshold. For example, the user 102 may be moving the paretic leg 104 (or the prosthetic leg 320) from a stance phase towards a swing phase to initiate or continue a walking movement sequence. For example, the orientation threshold may be anywhere in the range of about 5 degrees to about 20 degrees off of a vertical axis.

The control computing device 144 may further determine the angular velocity for the thigh portion 108 (or the thigh section 322) based on the received kinematic data. For example, the device 144 may parse the angular velocity data from the received kinematic data. The control computing device 144 may further determine that the angular velocity for the thigh portion 108 of the paretic leg 104 (or thigh section 322 of the prosthetic leg 320) satisfies an angular velocity threshold. For example, the determination may be based on the received kinematic data for the paretic leg 104 (or the prosthetic leg 320). For example, the control computing device 144 may compare the angular velocity of the thigh portion 108 (or the thigh section 322) to the angular velocity threshold. The control computing device 144 may determine that the angular velocity of the thigh portion 108 (or the thigh section 322) satisfies the angular velocity threshold if the angular velocity is one of greater than or greater than or equal to the angular velocity threshold. In other examples, assistance may be provided based on the angular velocity not satisfying the angular velocity threshold and the control computing device 144 may determine that the angular velocity does not satisfy the angular velocity threshold based on the angular velocity being greater than or greater than or equal to the angular velocity threshold. For example, the angular velocity threshold may be anywhere in the range of about 15 degrees per second to about 50 degrees per second.

At 530, at least one of electrical stimuli or motorized assistance may be provided to the paretic leg 104 (or the prosthetic leg 320). For example, the control computing device 144 or another computing device may cause the electrical stimuli and/or the motorized assistance to be provided to the paretic leg 104 (or the prosthetic leg 320). For example, the control computing device 144 may send a signal to one or more of the motors 128, 152 or 328, 352 to provide motorized assistance and/or to the electrodes 130A-B, 132 or the pulse generator 202 to provide electrical stimuli to the paretic leg 104 (or the remaining portion of thigh of the amputated leg). For example, the electrical stimuli and/or motorized assistance may be provided based on the angle of orientation of the portion (e.g., the thigh portion 108 or thigh section 322) of the paretic leg 104 (or the prosthetic leg 320) satisfying the orientation threshold. For example, the electrical stimuli and/or motorized assistance may be provided further based on the angular velocity of the portion (e.g., the thigh portion 108 or the thigh section 322) of the paretic leg 104 (or the prosthetic leg 320) satisfying the angular velocity threshold.

For example, the motor 128 or 328 may receive the signal from the control computing device 144 and may provide motorized assistance by causing the shank section 124 of the brace 120 (or shank section 324 of the prosthetic leg 320) to rotate with respect to the thigh section 122 of the brace 120 (or thigh section 322 of the prosthetic leg 320). For example, the motor 152 or 352 may receive the signal from the control computing device 144 and may provide motorized assistance by causing the thigh section 122 of the brace 120 (or thigh section 322 of the prosthetic leg 320) to rotate with respect to the hip section 150 of the brace 120 (or hip section 350 of the prosthetic leg 320). For example, the electrodes 130A-B, 132 or the pulse generator 202 may receive the signal from the control computing device 144 and provide electrical stimuli to portions of the paretic leg 104 (or the remaining portion of thigh of the amputated leg) by sending electrical pulses into the muscles of the paretic leg 104 (or the remaining portions of thigh of the amputated leg). For example, the signal may indicate which portions of the paretic leg 104 and/or which electrodes to activate to provide electrical stimuli to the paretic leg 104 (or the remaining portion of thigh of the amputated leg 320).

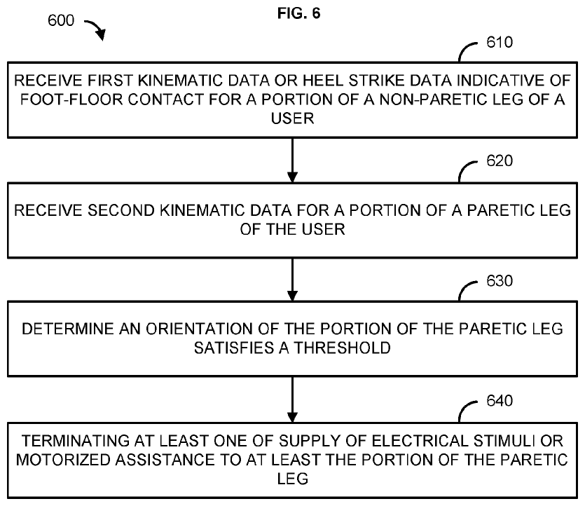

FIG. 6 shows an example method 600 for terminating or reducing assistance for leg movement. Referring to FIGS. 1-3 and 6, the method 600 may be completed by one or more of the control computing device 144, the brace 120 (or prosthetic leg 320), and/or the pulse generator 202. At 610, first kinematic data or heel-strike data indicative of foot-floor contact for a portion of a non-paretic leg 106 of a user 102 may be received. For example, the portion of the non-paretic leg 106 may be the foot 118. For example, the first kinematic data may have been received from one or more sensors 140-142 (e.g., the heel-strike sensor 142) receiving data about the foot 118 of the non-paretic leg 106. For example, the first kinematic data may be received by the control computing device 144 from the one or more of the sensors 140-142 (e.g., the heel-strike sensor 142). For example, the first kinematic data may be received from the foot sensor 140. In this example, the first kinematic data may comprise one or more of orientation of the foot 118 and/or angular velocity of the foot 118 and may indicate foot-floor contact for the foot 118 of the non-paretic leg 106 of the user 102. For example, foot-floor contact may indicate that all or at least a portion of the foot 118 is in contact with a floor surface. For example, the portion of the foot 118 may comprise the heel of the foot 118, and the foot-floor contact may indicate that the heel of the foot 118 is in contact with the floor surface. For example, the first kinematic data may comprise a calculated estimate of the center of mass of the body of the user 102.

At 620, second kinematic data for a portion of a paretic leg 104 (or the prosthetic leg 320) of the user 102 may be received. For example, the portion of the paretic leg 104 (or the prosthetic leg 320) may be the thigh portion 108 (or the thigh section 322) or the shank portion 110 (or the shank section 324). For example, the second kinematic data may be received by the control computing device 144 from one or more sensors 134-138 or 334-338 (e.g., the thigh sensor 134 or 334 or the shank sensor 136 or 336) receiving data about the particular portion of the paretic leg 104 (or the prosthetic leg 320). For example, the second kinematic data comprises one or more of a position of the portion of the paretic leg 104 (or the prosthetic leg 320), an angular velocity of the portion of the paretic leg 104 (or the prosthetic leg 320), an orientation of the portion of the paretic leg 104 (or the prosthetic leg 320), muscle activity data for the portion of the paretic leg 104 (or the prosthetic leg 320), and/or an acceleration of the portion of the paretic leg 104 (or the prosthetic leg 320). For example, the second kinematic data comprises an angle of orientation of at least one of the thigh portion 108 or shank portion 110 of the paretic leg 104 (or the prosthetic leg 320). For example, the second kinematic data may indicate that the paretic leg 104 (or the prosthetic leg 320) is in an extended position. For example, the second kinematic data may comprise a calculated estimate of the center of mass of the body of the user 102.

For example, the second kinematic data may comprise orientation data (e.g., an angle of orientation) for the thigh portion 108 of the paretic leg 104 (or the thigh section 322 of the prosthetic leg 320). For example, the angle of orientation for the thigh portion 108 may be determined based on the angle between the longitudinal axis α of the thigh portion 108 and one of a vertical axis and a horizontal axis. For example, the second kinematic data may further comprise acceleration data for the thigh portion 108 of the paretic leg 104 (or the thigh section 322 of the prosthetic leg 320), velocity data (e.g., angular velocity data) for the thigh portion 108 of the paretic leg 104 (or the thigh section 322 of the prosthetic leg 320), and/or position data for the thigh portion 108 of the paretic leg 104 (or the thigh section 322 of the prosthetic leg 320).

The control computing device 144 may determine the orientation (e.g., the angle of orientation) for the thigh portion 108 or the shank portion 110 based on the second kinematic data. For example, the device 144 may parse the angle of orientation for the thigh portion 108 or the shank portion 110 from the orientation data of the received second kinematic data. At 630, a determination may be made that the angle of orientation for the portion of the paretic leg 104 (or prosthetic leg 320) satisfies an orientation threshold. For example, the determination may be based on the received second kinematic data for the paretic leg 104 (or the prosthetic leg 320). The determination may be made by the control computing device 144 or another computing device. For example, the orientation threshold may comprise an angle relative to a vertical axis or a horizontal axis.

For example, the control computing device 144 may compare the angle of orientation of the thigh portion 108 (or the thigh section 322 of the prosthetic leg 320) to the orientation threshold to determine if the angle of orientation of the thigh portion 108 (or the thigh section 322 of the prosthetic leg 320) satisfies the orientation threshold. The angle of orientation of the thigh portion 108 (or the thigh section 322 of the prosthetic leg 320) satisfies the orientation threshold if the angle of orientation is one of less than or less than or equal to the orientation threshold. In other examples, assistance may be terminated or reduced based on the angle of orientation not satisfying the threshold and the control computing device 144 may determine that the angle of orientation does not satisfy the orientation threshold based on the angle of orientation being less than or less than or equal to the orientation threshold. For example, the control computing device 144 may be evaluating the sensors (e.g., the thigh sensor 134 or 334 or the shank sensor 136 or 336) to determine if the user 102 is transitioning from a walking motion to a quiet stance or stopped position. For example, the orientation threshold may be anywhere in the range of about 0 degrees to about −15 degrees off of a vertical axis.

In another example, rather than the angle of orientation of the thigh portion 108 (or the thigh section 322 of the prosthetic leg 320), the angle of orientation of the shank portion 110 (or the shank section 324 of the prosthetic leg 320) may be evaluated against the orientation threshold. For example, the angle of orientation of the shank portion 110 (or the shank section 324 of the prosthetic leg 320) may be determined as the angle between the longitudinal axis β of the shank portion 110 (or the shank section 324 of the prosthetic leg 320) and one of a vertical axis and a horizontal axis. In this other example, at 630, the control computing device 144 may compare the angle of orientation of the shank portion 110 (or the shank section 324 of the prosthetic leg 320) to the orientation threshold to determine if the angle of orientation satisfies the orientation threshold. The angle of orientation of the shank portion 110 (or the shank section 324 of the prosthetic leg 320) satisfies the threshold if the angle of orientation is one of less than or less than or equal to the orientation threshold. In other examples, assistance may be terminated or reduced based on the angle of orientation not satisfying the threshold and the control computing device 144 may determine that the angle of orientation does not satisfy the orientation threshold based on the angle of orientation being less than or less than or equal to the orientation threshold.

At 640, the provision of at least one of electrical stimuli or motorized assistance may be terminated or reduced at the paretic leg 104 (or the prosthetic leg 320). Based on the control computing device 144 determining that the user 102 is likely transitioning from a walking phase to a quiet stance phase, all electrical or motorized assistance may be terminated or reduced, as it is not needed, or is needed less, by the user when in a quiet stance (e.g., standing still). For example, the control computing device 144 or another computing device may cause the electrical stimuli and/or the motorized assistance to be terminated or reduced to the paretic leg 104 (or the prosthetic leg 320). Determining whether to terminate or reduce one or both of electrical stimuli or motorized assistance may be based on whether one or both are being provided to the paretic leg 104 (or the prosthetic leg 320) of the user. For example, if only motorized assistance is being provided to the paretic leg 104 (or the prosthetic leg 320) of the user 102, then only motorized assistance needs to be terminated or reduced. Likewise if only electrical stimuli is being provided to the paretic leg 104 (or the prosthetic leg 320).

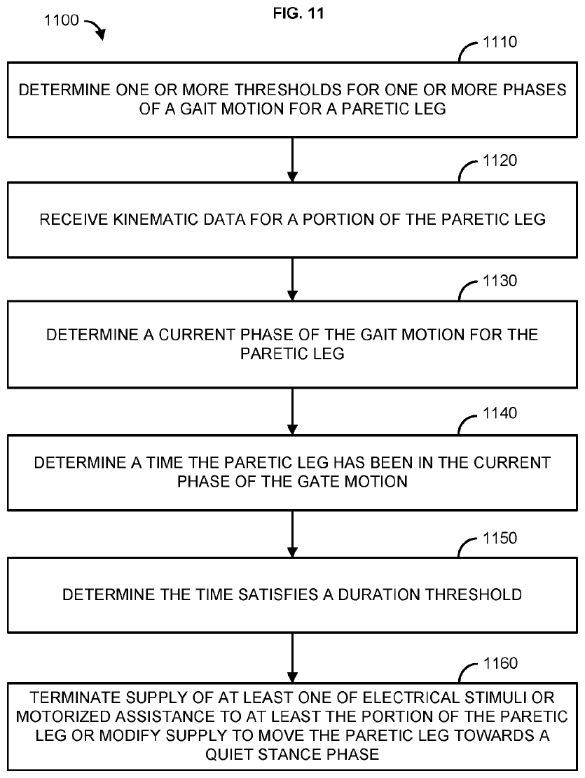

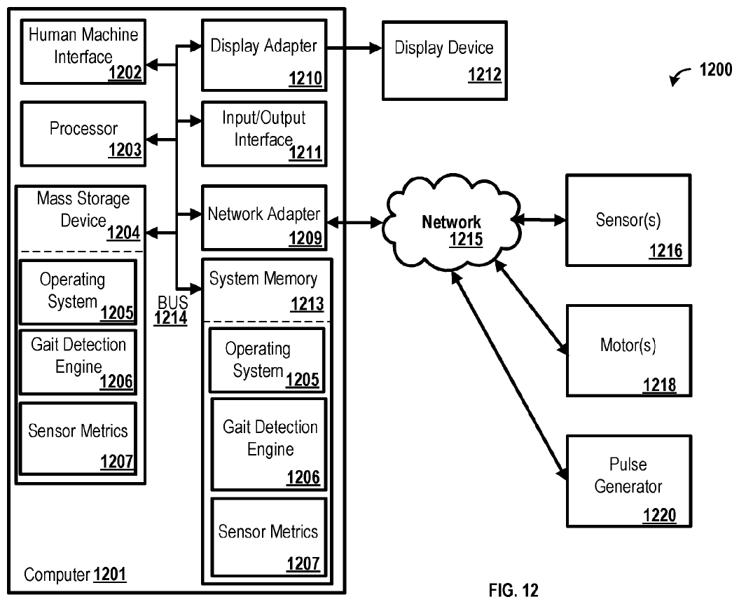

For example, the control computing device 144 may send a signal to one or more of the motors 128, 152 or 328, 352 to terminate or reduce motorized assistance and/or to the electrodes 130A-B, 132 or the pulse generator 202 to terminate (e.g., stop) or reduce providing electrical stimuli to the paretic leg 104 (or the prosthetic leg 320). For example, the electrical stimuli and/or motorized assistance may be terminated or reduced based on the angle of orientation of the portion (e.g., the thigh portion 108 or thigh section 322 or the shank portion 110 or shank section 324) of the paretic leg 104 (or the prosthetic leg 320) satisfying the orientation threshold and based on the first kinematic data or the heel-strike data for the portion of the non-paretic leg 106 indicating foot floor contact for the portion (e.g., foot 118) non-paretic leg 106. In another example, the electrical stimuli and/or motorized assistance may be terminated or reduced based on the time it took to complete a current phase of the gait cycle satisfying a duration threshold as described in 1110-1160 of FIG. 11 below.

For example, one or more of the motors 128, 152 or 328, 352 may receive the signal from the control computing device 144 and may terminate or reduce motorized assistance. For example, the electrodes 130A-B, 132 or the pulse generator 202 may receive the signal from the control computing device 144 and may terminate or reduce electrical stimuli to all portions of the paretic leg 104 (or the remaining portion of the amputated leg).

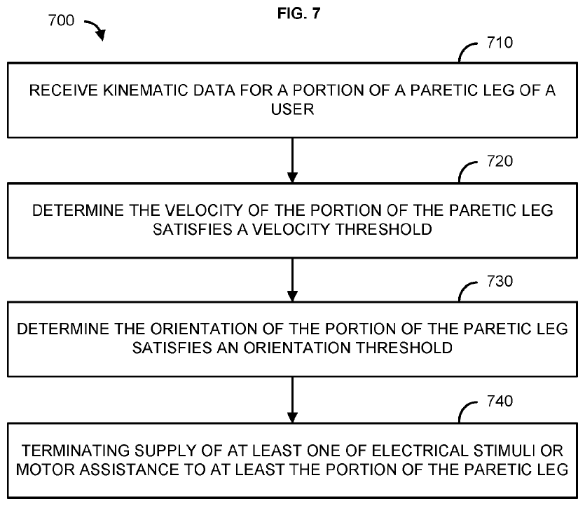

FIG. 7 shows an example method 700 for terminating or reducing assistance for leg movement. Referring to FIGS. 1-3 and 7, the method 700 may be completed by one or more of the control computing device 144, the brace 120 (or prosthetic leg 320), and/or the pulse generator 202. At 710, kinematic data for a portion of a paretic leg 104 (or prosthetic leg 320) may be received. For example, the portion of the paretic leg 104 (or the prosthetic leg 320) may be the thigh portion 108 (or the thigh section 322). In other examples, the portion of the paretic leg 104 (or the prosthetic leg 320) may be the shank portion 110 (or the shank section 324). For example, the kinematic data may have been received from one or more sensors 134-138 or 334-338 (e.g., the thigh sensor 134 or 334) receiving data about the paretic leg 104 (or the prosthetic leg 320). For example, the kinematic data may be received by the control computing device 144 from one or more of the sensors 134-138 or 334-338 (e.g., the thigh sensor 134 or 334). For example, a first portion of the kinematic data may be received from a first sensor coupled to the portion of the paretic leg 104 (or the prosthetic leg 320) and a second portion of the kinematic data may be received from a second sensor coupled to the portion of the paretic leg 104 (or the prosthetic leg 320).

For example, the kinematic data may comprise orientation data (e.g., an angle of orientation) for the thigh portion 108 of the paretic leg 104 (or the thigh section 322 of the prosthetic leg 320) and velocity data (e.g., angular velocity data) for the thigh portion 108 (or the thigh section 322 of the prosthetic leg 320). For example, the angle of orientation for the thigh portion 108 (or the thigh section 322 of the prosthetic leg 320) may be determined based on the angle between the longitudinal axis α of the thigh portion 108 (or the thigh section 322) and one of a vertical axis and a horizontal axis. For example, the kinematic data may further comprise acceleration data for the thigh portion 108 of the paretic leg 104 (or the thigh section 322 of the prosthetic leg 320) and/or position data for the thigh portion 108 of the paretic leg 104 (or the thigh section 322 of the prosthetic leg 320). For example, the kinematic data may further comprise muscle activity data for the thigh portion 108 of the paretic leg 104 (or the remaining portion of the amputated leg). For example, the kinematic data may also comprise a calculated estimate of the center of mass of the body of the user 102.

The control computing device 144 may determine the angular velocity for the thigh portion 108 (or the thigh section 322) based on the received kinematic data. For example, the device 144 may parse the velocity data from the received kinematic data from the sensor 134 or 334. For example, the angular velocity may be the angular velocity of the thigh portion 108 (or the thigh section 322) at a particular point in time. In other examples, the velocity represents the change in angular velocity between two time periods of received kinematic data from the sensor (e.g., the sensor 134 or 334). For example, the control computing device 144 may receive a first angular velocity for a first portion (e.g., the thigh portion 108 or thigh section 322 or the shank portion 110 or shank section 324) at a first time from a sensor (e.g., the sensor 134 or 136 or 334 or 336). The control computing device 144 may receive a second angular velocity for the particular portion of the paretic leg 104 (or the prosthetic leg 320) at a second time from the particular sensor. The control computing device 144 may determine a change in angular velocity for the particular portion of the paretic leg 104. For example, the change in angular velocity may be determined as the difference between the first angular velocity and the second angular velocity. The control computing device 144 may then compare the change in angular velocity to the angular velocity threshold as described below in 720.

At 720, a determination may be made that the angular velocity for the thigh portion 108 of the paretic leg 104 (or the thigh section 322 of the prosthetic leg 320) satisfies an angular velocity threshold. The determination may be made by the control computing device 144 or another computing device. For example, the determination may be based on the received kinematic data for the paretic leg 104 (or the prosthetic leg 320). For example, the control computing device 144 may compare the angular velocity of the thigh portion 108 (or the thigh section 322) to the angular velocity threshold. The device 144 may determine that the angular velocity of the thigh portion 108 (or the thigh section 322) satisfies the angular velocity threshold if the angular velocity is one of less than or less than or equal to the angular velocity threshold. In other examples, termination or reduction of assistance to the paretic leg 104 (or the prosthetic leg 320) may be based on the angular velocity not satisfying the angular velocity threshold and the control computing device 144 may determine that the angular velocity does not satisfy the angular velocity threshold based on the angular velocity being less than or less than or equal to the angular velocity threshold. For example, the angular velocity threshold may be anywhere in the range of about 0 degrees per second to about 50 degrees per second.

The control computing device 144 may determine the orientation (e.g., the angle of orientation) for the thigh portion 108 (or the thigh section 322) based on the kinematic data. For example, the device 144 may parse the angle of orientation from the orientation data of the received kinematic data from the thigh sensor 134 or 334. At 730, a determination may be made that the orientation (e.g., the angle of orientation) for the portion (e.g., the thigh portion 108) of the paretic leg 104 (or the thigh section 322 of the prosthetic leg 320) satisfies an orientation threshold. For example, the determination may be based on the received kinematic data for the paretic leg 104 (or the prosthetic leg 320). The determination may be made by the control computing device 144 or another computing device. For example, the portion of the paretic leg 104 (or the prosthetic leg 320) may be the thigh portion 108 (or the thigh section 322). In other examples, the portion of the paretic leg 104 (or the prosthetic leg 320) may be the shank portion 110 (or the shank section 324). For example, the control computing device 144 may compare the angle of orientation of the thigh portion 108 (or the thigh section 322) to the orientation threshold to determine if the angle of orientation of the thigh portion 108 (or the thigh section 322) satisfies the orientation threshold. The angle of orientation of the thigh portion 108 (or the thigh section 322) satisfies the threshold if the angle of orientation is one of less than or less than or equal to the orientation threshold. In other examples, termination or reduction of assistance to the paretic leg 104 (or the prosthetic leg 320) may be based on the angle of orientation not satisfying the threshold and the control computing device 144 may determine that the angle of orientation does not satisfy the orientation threshold based on the angle of orientation being less than or less than or equal to the orientation threshold. For example, the orientation threshold may be anywhere in the range of about 5 degrees to about −20 degrees off of a vertical axis.

For example, the orientation data of the thigh sensor 134 or 334 may indicate that the paretic leg 104 (or the prosthetic leg 320) is in a midswing phase or terminal swing phase of a gait cycle. The control computing device 144 may be evaluating the angular velocity of the paretic leg 104 (or the prosthetic leg 320)(e.g., via the kinematic data from the thigh sensor 134 or 334) to determine if the paretic leg 104 (or the prosthetic leg 320) is maintaining a speed to continue walking or if the paretic leg 104 is slowing down, indicating that the user 102 may be transitioning from walking to a quiet stance (e.g., standing still), for which additional motorized assistance or electrical stimuli will not be needed. Based on the angular velocity satisfying the threshold (e.g., equal to or below the angular velocity threshold), the control computing device 144 may determine that the user 102 is transitioning to a quiet stance.

At 740, the provision of at least one of electrical stimuli or motorized assistance may be terminated or reduced at the paretic leg 104 (or the prosthetic leg 320). Based on the control computing device 144 determining that the user 102 is likely transitioning from a walking phase to a quiet stance phase, all electrical or motorized assistance may be terminated or reduced, as it is not needed by the user 102 (or is needed to a lesser extent) when in a quiet stance (e.g., standing still). For example, the control computing device 144 or another computing device may cause the electrical stimuli and/or the motorized assistance to be terminated or reduced to the paretic leg 104 (or the prosthetic leg 320). Determining whether to terminate or reduce one or both of electrical stimuli or motorized assistance may be based on whether one or both are being provided to the paretic leg 104 (or the prosthetic leg 320) of the user 102. For example, if only motorized assistance is being provided to the paretic leg 104 (or the prosthetic leg 320) of the user 102, then only motorized assistance needs to be terminated or reduced. Likewise if only electrical stimuli is being provided to the paretic leg 104 (or the remaining portion of the thigh of the amputated leg), only electrical stimuli needs to be terminated or reduced.

For example, the control computing device 144 may send a signal to one or more of the motors 128, 152 or 328, 352 to terminate or reduce motorized assistance and/or to the electrodes 130A-B, 132 or the pulse generator 202 to terminate providing or reduce the provision of electrical stimuli to the paretic leg 104 (or the prosthetic leg 320). For example, the electrical stimuli and/or motorized assistance may be terminated or reduced based on the orientation (e.g., angle of orientation) of the portion (e.g., the thigh portion 108 or the thigh section 322 or the shank portion 110 or the shank section 324) of the paretic leg 104 (or the prosthetic leg 320) satisfying the orientation threshold and based on the angular velocity of the portion (e.g., the thigh portion 108 or the thigh section 322 or the shank portion 110 or the shank section 324) of the paretic leg 104 (or the prosthetic leg 320) satisfying the angular velocity threshold.

For example, terminating or reducing the electrical stimuli and/or motorized assistance may be based on the portion of the paretic leg 104 (or the prosthetic leg 320) being in a second position (or second angle of orientation). For example, second kinematic data may be received. The second kinematic data may be received by the control computing device 144 or another computing device. The second kinematic data may indicate that the portion (e.g., the thigh portion 108 or the thigh section 322 or the shank portion 110 or the shank section 324) is in a second position of a gait cycle. For example, the second position of the gait cycle may comprise the terminal swing phase of the gait cycle. For example, the determination may be made by comparing the second angle of orientation of the portion of the paretic leg 104 (or the prosthetic leg 320) to a second orientation threshold to determine that the second angle of orientation satisfies the second threshold. For example, terminating or reducing the electrical stimuli and/or motorized assistance for the paretic leg 104 (or the prosthetic leg 320) may be based on the second angle of orientation satisfying the second threshold and the angular velocity satisfying the angular velocity threshold. For example, the electrical stimuli and/or motorized assistance may be terminated or reduced based on the time it took to complete a current phase of the gait cycle satisfying a duration threshold as described in 1110-1160 of FIG. 11 below.

For example, one or more of the motors 128, 152 or 328, 352 may receive the signal from the control computing device 144 and may terminate or reduce the motorized assistance. For example, the electrodes 130A-B, 132 or the pulse generator 202 may receive the signal from the control computing device 144 and may terminate or reduce the amount of electrical stimuli to all portions or particular portions of the paretic leg 104 (or the remaining portion of the thigh of the amputated leg).

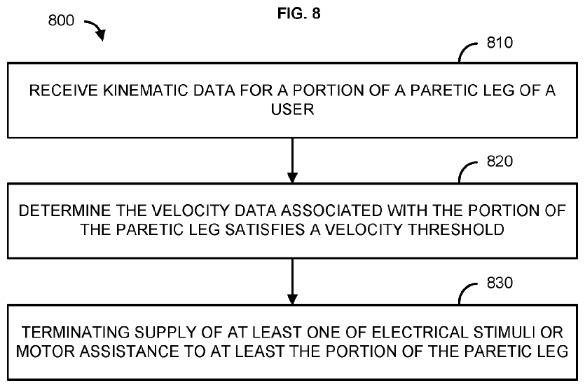

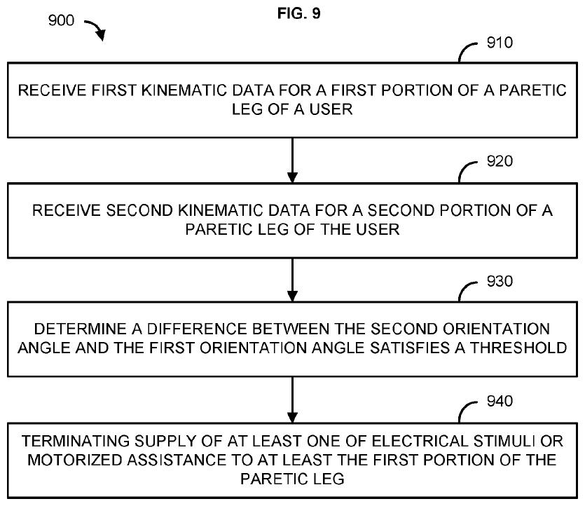

FIG. 8 shows an example method 800 for terminating or reducing assistance for leg movement. Referring to FIGS. 1-3 and 8, the method 800 may be completed by one or more of the control computing device 144, the brace 120 (or prosthetic leg 320), and/or the pulse generator 202. At 810, kinematic data for a portion of a paretic leg 104 (or the prosthetic leg 320) may be received. For example, the portion of the paretic leg 104 may be the thigh portion 108 (or the thigh section 322 of the prosthetic leg 320). In other examples, the portion of the paretic leg 104 may be the shank portion 110 (or the shank section 324 of the prosthetic leg 320). For example, the kinematic data may have been received from one or more sensors 134-138 or 334-338 (e.g., the thigh sensor 134 or 334) receiving data about the paretic leg 104 (or the prosthetic leg 320). For example, the kinematic data may be received by the control computing device 144 from one or more of the sensors 134-138 or 334-338 (e.g., the thigh sensor 134 or 334).

For example, the kinematic data may comprise velocity data (e.g., angular velocity data) and orientation data (e.g., an angle of orientation) for the portion (e.g., the thigh portion 108) of the paretic leg 104 (or the thigh section 322 of the prosthetic leg 320). For example, a first portion of the kinematic data (e.g., the angular velocity data) may be received from a first sensor coupled to the portion of the paretic leg 104 (or the prosthetic leg 320) and a second portion of the kinematic data (e.g., the position data) may be received from a second sensor coupled to the portion of the paretic leg 104 (or the prosthetic leg 320). The kinematic data may further comprise acceleration data for the portion of the paretic leg 104 (or prosthetic leg 320). For example, the kinematic data may also comprise a calculated estimate of the center of mass of the body of the user 102.

For example, the position of the paretic leg 104 may be determined based on an angle of orientation for the portion (e.g., the thigh portion 108 322) of the paretic leg 104 (or the prosthetic leg 320). For example, the angle of orientation for the thigh portion 108 (or the thigh section 322) may be determined based on the angle between the longitudinal axis α of the thigh portion 108 (or the thigh section 322) and one of a vertical axis and a horizontal axis. For example, the position of the portion of the paretic leg 104 (or the prosthetic leg 320) may further be determined based on the calculated estimate of the center of mass of the body of the user 102.

For example, the position of the portion (e.g., the thigh portion 108 or the thigh section 322) of the paretic leg 104 may be determined based on the orientation (e.g., the angle of orientation) for the portion (e.g., the thigh portion 108 or thigh section 322) of the paretic leg 104 (or the prosthetic leg 320) satisfying an orientation threshold. For example, the orientation threshold may be an angular range and the angle of orientation may satisfy the orientation threshold if the angle of orientation for the portion of the paretic leg 104 (or the prosthetic leg 320) is within the angular range. For example, the angular range of the orientation threshold may comprise a lower angle of orientation boundary and an upper angle or orientation boundary. For example, the angle of orientation satisfying the threshold may comprise the angle of orientation being between the lower angle of orientation and the upper angle of orientation.

For example, the determination that the angle of orientation satisfies and orientation threshold may be based on the received kinematic data for the paretic leg 104 (or the prosthetic leg 320). The determination may be made by the control computing device 144 or another computing device. For example, the portion of the paretic leg 104 may be the thigh portion 108 (or the thigh section 322 of the prosthetic leg 320). In other examples, the portion of the paretic leg 104 may be the shank portion 110 (or the shank section 324 of the prosthetic leg 320). For example, the control computing device 144 may compare the angle of orientation of the thigh portion 108 (or the thigh section 322) to the angular range of the orientation threshold to determine if the angle of orientation of the thigh portion 108 (or the thigh section 322) satisfies (e.g., is within the angular range of) the orientation threshold. Based on the angle of orientation being within the angular range and satisfying the orientation threshold, the control computing device 144 may determine the position of the portion of the paretic leg 104 (or the prosthetic leg 320). For example, the device 144 may determine the current phase of the gait cycle for the paretic leg 104 (or the prosthetic leg 320) based on the position of the paretic leg (or the prosthetic leg 320). For example, the device 144 may determine that the paretic leg 104 (or the prosthetic leg 320) is in the mid-stance phase of the gait cycle based on the position of the portion (e.g., thigh portion 108 or thigh section 322) of the paretic leg 104 (or the prosthetic leg 320). For example, the angular range of the orientation threshold may be a range of about −15 degrees to about 15 degrees off of a vertical axis.

The control computing device 144 may determine the angular velocity for the portion (e.g., the thigh portion 108 or the thigh section 322) of the paretic leg 104 (or the prosthetic leg 320) based on the received kinematic data. For example, the device 144 may parse the velocity data (e.g., the angular velocity data) from the received kinematic data from the sensor 134 or 334. For example, the angular velocity may be the angular velocity of the thigh portion 108 (or the thigh section 322 of the prosthetic leg 320) at a particular point in time. At 820, a determination may be made that the angular velocity for the portion (e.g., the thigh portion 108 or the thigh section 322) of the paretic leg 104 (or the prosthetic leg 320) satisfies an angular velocity threshold. The determination may be made by the control computing device 144 or another computing device. For example, the determination may be based on the received kinematic data for the paretic leg 104 (or the prosthetic leg 320). For example, the control computing device 144 may compare the angular velocity of the portion (e.g., the thigh portion 108 or the thigh section 322) to the angular velocity threshold. The device 144 may determine that the angular velocity of the portion of the paretic leg 104 (or the prosthetic leg 320) satisfies the angular velocity threshold if the angular velocity is one of less than or less than or equal to the angular velocity threshold. In other examples, termination or reduction of assistance to the paretic leg 104 (or the prosthetic leg 320) may be based on the angular velocity not satisfying the angular velocity threshold and the control computing device 144 may determine that the angular velocity does not satisfy the angular velocity threshold based on the angular velocity being less than or less than or equal to the angular velocity threshold. For example, the angular velocity threshold may be anywhere in the range of about 0 degrees per second to about 50 degrees per second.

At 830, the provision of at least one of electrical stimuli or motorized assistance may be terminated or reduced at the paretic leg 104 (or the prosthetic leg 320). Based on the control computing device 144 determining that the user 102 is likely transitioning from a walking phase to a quiet stance phase, all electrical or motorized assistance may be terminated or reduced, as it is not needed by the user 102 (or needed to a lesser extent) when in a quiet stance (e.g., standing still). For example, the control computing device 144 or another computing device may cause the electrical stimuli and/or the motorized assistance to be terminated or reduced to the paretic leg 104 (or the prosthetic leg 320). Determining whether to terminate or reduce one or both of electrical stimuli or motorized assistance may be based on whether one or both are being provided to the paretic leg 104 (or the prosthetic leg 320) of the user 102. For example, if only motorized assistance is being provided to the paretic leg 104 (or the prosthetic leg 320) of the user 102, then only motorized assistance needs to be terminated or reduced. Likewise if only electrical stimuli is being provided to the paretic leg 104 (or the remaining portion of the thigh of the amputated leg), only electrical stimuli needs to be terminated or reduced.

For example, the control computing device 144 may send a signal to one or more of the motors 128, 152 or 328, 352 to terminate or reduce motorized assistance and/or to the electrodes 130A-B, 132 or the pulse generator 202 to terminate providing or reduce the provision of electrical stimuli to the paretic leg 104 (or the prosthetic leg 320). For example, the electrical stimuli and/or motorized assistance may be terminated or reduced based on the position (e.g., the angle of orientation being within the angular range of the orientation threshold) of the portion (e.g., the thigh portion 108 or thigh section 322) of the paretic leg 104 (or the prosthetic leg 320) and based on the angular velocity of the portion (e.g., the thigh portion 108 or the thigh section 322 or the shank portion 110 or the shank section 324) of the paretic leg 104 (or the prosthetic leg 320) satisfying the angular velocity threshold. For example, the electrical stimuli and/or motorized assistance may be terminated or reduced further based on the calculated estimate of the center of mass of the body of the user 102. For example, the electrical stimuli and/or motorized assistance may be terminated or reduced based on the time it took to complete a current phase of the gait cycle satisfying a duration threshold as described in 1110-1160 of FIG. 11 below.

For example, one or more of the motors 128, 152 or 328, 352 may receive the signal from the control computing device 144 and may terminate or reduce motorized assistance. For example, the electrodes 130A-B, 132 or the pulse generator 202 may receive the signal from the control computing device 144 and may terminate or reduce electrical stimuli to all portions of the paretic leg 104 (or the remaining portion of the thigh of the amputated leg).

FIG. 9 shows an example method 900 for terminating or reducing assistance for leg movement. Referring to FIGS. 1-3 and 9, the method 900 may be completed by one or more of the control computing device 144, the brace 120 (or prosthetic leg 320), and/or the pulse generator 202. At 910, first kinematic data for a first portion of a paretic leg 104 (or the prosthetic leg 320) may be received. For example, the first portion of the paretic leg 104 may be the thigh portion 108 (or the thigh section 322 of the prosthetic leg 320). For example, the first kinematic data may be received by the control computing device 144 from the thigh sensor 134 or 334. For example, the first kinematic data may comprise first orientation data (e.g., a first angle of orientation) for the thigh portion 108 of the paretic leg 104 (or the thigh section 322 of the prosthetic leg 320). For example, the angle of orientation for the thigh portion 108 (or the thigh section 322) may be determined based on the angle between the longitudinal axis α of the thigh portion 108 (or the thigh section 322) and one of a vertical axis and a horizontal axis. For example, the first kinematic data may further comprise velocity data (e.g., angular velocity data) for the thigh portion 108 (or the thigh section 322), acceleration data for the thigh portion 108 (or the thigh section 322), and/or position data for the thigh portion 108 (or the thigh section 322). For example, the first kinematic data may further comprise muscle activity data for the thigh portion 108 (or the remainder of the thigh of the amputated leg) of the paretic leg 104. For example, the first kinematic data may also comprise a calculated estimate of the center of mass of the body of the user 102.

At 920, second kinematic data for a second portion of a paretic leg 104 (or the prosthetic leg 320) may be received. For example, the second portion of the paretic leg 104 (or the prosthetic leg 320) may be the shank portion 110 (or shank section 322). For example, the second kinematic data may be received by the control computing device 144 from the shank sensor 136 or 336. For example, the second kinematic data may comprise second orientation data (e.g., a second angle of orientation) for the shank portion 110 of the paretic leg 104 (or the shank section 324 of the prosthetic leg 320). For example, the angle of orientation for the shank portion 110 (or the shank section 324) may be determined based on the angle between the longitudinal axis β of the shank portion 110 (or the shank section 324) and one of a vertical axis and a horizontal axis. For example, the second kinematic data may further comprise velocity data (e.g., angular velocity data) for the shank portion 110 (or the shank section 324), acceleration data for the shank portion 110 (or the shank section 324), and/or position data for the shank portion 110 (or the shank section 324). For example, the second kinematic data may further comprise muscle activity data for the shank portion 110 of the paretic leg 104. For example, the second kinematic data may also comprise a calculated estimate of the center of mass of the body of the user 102.

The control computing device 144 may determine the difference between the second angle of orientation and the first angle of orientation. At 930, a determination may be made that the difference between the second angle of orientation and the first angle of orientation satisfies a relative orientation threshold. For example, the determination may be based on the received kinematic data for the paretic leg 104 (or the prosthetic leg 320). The determination may be made by the control computing device 144 or another computing device. For example, the control computing device 144 may compare the difference between the second angle of orientation (for the shank portion 110 or the shank section 324) and the first angle of orientation (for the thigh portion 108 or the thigh section 322) to the orientation threshold to determine if the difference satisfies the orientation threshold. The difference satisfies the orientation threshold if the difference is one of less than or less than or equal to the orientation threshold. In other examples, termination or reduction of assistance to the paretic leg 104 (or the prosthetic leg 320) may be based on the difference not satisfying the threshold and the control computing device 144 may determine that the difference does not satisfy the orientation threshold based on the difference being less than or less than or equal to the orientation threshold. For example, the orientation threshold may be anywhere in the range of about 0 degrees to about −15 degrees.

At 940, the provision of at least one of electrical stimuli or motorized assistance may be terminated or reduced at the paretic leg 104 (or the prosthetic leg 320). Based on the control computing device 144 determining that the user 102 is likely transitioning to a quiet stance phase, all electrical or motorized assistance may be terminated or reduced, as it is not needed by the user 102 (or is needed to a lesser extent) when in a quiet stance (e.g., standing still). For example, the control computing device 144 or another computing device may cause the electrical stimuli and/or the motorized assistance to be terminated or reduced to the paretic leg 104 (or the prosthetic leg 320). Determining whether to terminate or reduce one or both of electrical stimuli or motorized assistance may be based on whether one or both are being provided to the paretic leg 104 (or the prosthetic leg 320) of the user 102. For example, if only motorized assistance is being provided to the paretic leg 104 (or the prosthetic leg 320) of the user 102, then only motorized assistance needs to be terminated or reduced. Likewise if only electrical stimuli is being provided to the paretic leg 104 (or the remaining portion of the thigh of the amputated leg), only electrical stimuli needs to be terminated or reduced.

For example, the control computing device 144 may send a signal to one or more of the motors 128, 152 or 328, 352 to terminate or reduce motorized assistance and/or to the electrodes 130A-B, 132 or the pulse generator 202 to terminate providing or reduce the provision of electrical stimuli to the paretic leg 104 (or the prosthetic leg 320). For example, the electrical stimuli and/or motorized assistance may be terminated or reduced based on the difference of the second angle of orientation and the first angle of orientation satisfying the orientation threshold. For example, the electrical stimuli and/or motorized assistance may be terminated or reduced based on the time it took to complete a current phase of the gait cycle satisfying a duration threshold as described in 1110-1160 of FIG. 11 below.

For example, one or more of the motors 128, 152 or 328, 352 may receive the signal from the control computing device 144 and may terminate or reduce motorized assistance. For example, the electrodes 130A-B, 132 or the pulse generator 202 may receive the signal from the control computing device 144 and may terminate or reduce electrical stimuli to all portions of the paretic leg 104 (or the remaining portion of the thigh of the amputated leg).

The method 900 may comprise the control computing device 144 determining, based on the received first kinematic data, the angular velocity of the thigh portion 108 of the paretic leg (or the thigh section 322 of the prosthetic leg 320). The control computing device 144 may compare the angular velocity of the thigh portion 108 (or the thigh section 322) to an angular velocity threshold to determine if the angular velocity of the thigh portion 108 (or the thigh section 322) satisfies the angular velocity threshold. For example, the control computing device 144 may determine that the angular velocity of the thigh portion 108 (or the thigh section 322) satisfies the angular velocity threshold if the angular velocity is one of less than or less than or equal to the angular velocity threshold. In other examples, termination or reduction of assistance to the paretic leg 104 (or the thigh section 322) may be based on the angular velocity not satisfying the angular velocity threshold and the control computing device 144 may determine that the angular velocity does not satisfy the angular velocity threshold based on the angular velocity being less than or less than or equal to the angular velocity threshold. For example, terminating or reducing the motorized assistance and/or electrical stimuli may be further based on the angular velocity of the thigh portion 108 (or the thigh section 322) satisfying the angular velocity threshold. For example, the angular velocity threshold may be anywhere in the range of about 0 degrees per second to about 100 degrees per second.

The method 900 may comprise the control computing device 144 determining a change or difference in velocity (e.g., difference in angular velocity) and comparing the difference in angular velocity to the angular velocity threshold. For example, the control computing device 144 may receive a first angular velocity for the first portion (e.g., the thigh portion 108 or thigh section 322) or the paretic leg 104 (or the prosthetic leg 320) at a first time from the thigh sensor 134 or 334. The control computing device 144 may receive a second angular velocity for the first portion of the paretic leg 104 (or the prosthetic leg 320) at a second time from the thigh sensor 134 or 334. The control computing device 144 may determine a change in angular velocity for the thigh portion 108 of the paretic leg 104 (or the thigh section 322 of the prosthetic leg 320). For example, the change in angular velocity may be determined as the difference between the first angular velocity and the second angular velocity.

The control computing device 144 may then compare the change in angular velocity to the angular velocity threshold. For example, the control computing device 144 may determine that the change in angular velocity of the thigh portion 108 (or the thigh section 322) satisfies the angular velocity threshold if the change in angular velocity is one of greater than or greater than or equal to the angular velocity threshold. In other examples, termination or reduction of assistance to the paretic leg 104 (or the prosthetic leg 320) may be based on the change in angular velocity not satisfying the angular velocity threshold and the control computing device 144 may determine that the change in angular velocity does not satisfy the angular velocity threshold based on the change in angular velocity being greater than or greater than or equal to the angular velocity threshold. For example, terminating or reducing the motorized assistance and/or electrical stimuli may be further based on the change in angular velocity of the thigh portion 108 (or the thigh section 322) satisfying the angular velocity threshold. For example, the angular velocity threshold may be anywhere in the range of about 0 degrees per second to about 80 degrees per second.

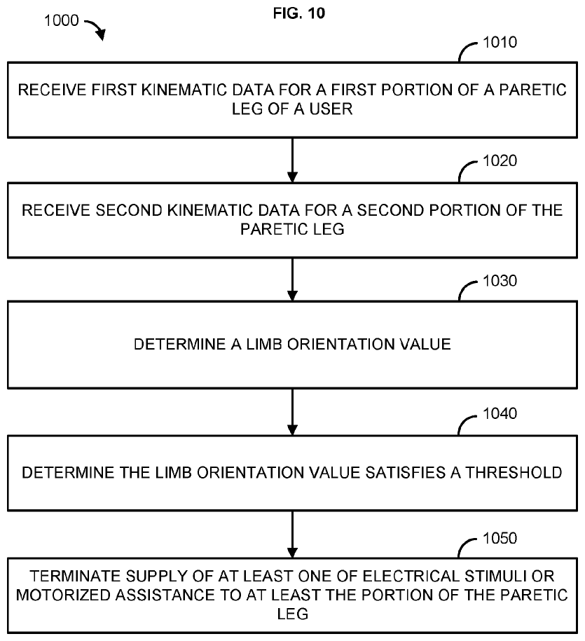

FIG. 10 shows an example method 1000 for terminating or reducing assistance for leg movement. Referring to FIGS. 1-3 and 10, the method 1000 may be completed by one or more of the control computing device 144, the brace 120 (or prosthetic leg 320), and/or the pulse generator 202. At 1010, first kinematic data for a first portion of a paretic leg 104 (or the prosthetic leg 320) may be received. For example, the first portion of the paretic leg 104 may be the thigh portion 108 (or the thigh section 322 of the prosthetic leg 320). For example, the first kinematic data may be received by the control computing device 144 from the thigh sensor 134 or 334. For example, the first kinematic data may comprise first orientation data (e.g., a first angle of orientation) for the thigh portion 108 of the paretic leg 104 (or the thigh section 322 of the prosthetic leg 320). For example, the angle of orientation for the thigh portion 108 (or the thigh section 322) may be determined based on the angle between the longitudinal axis α of the thigh portion 108 (or the thigh section 322) and one of a vertical axis and a horizontal axis. For example, the first kinematic data may further comprise angular velocity data for the thigh portion 108 (or the thigh section 322), acceleration data for the thigh portion 108 (or the thigh section 322), and/or position data for the thigh portion 108 (or the thigh section 322). For example, the first kinematic data may further comprise muscle activity data for the thigh portion 108 of the paretic leg 104 (or the remaining portion of the thigh of the amputated leg). For example, the first kinematic data may also comprise a calculated estimate of the center of mass of the body of the user 102.

At 1020, second kinematic data for a second portion of a paretic leg 104 (or the prosthetic leg 320) may be received. For example, the second portion of the paretic leg 104 may be the shank portion 110 (or the shank section 324 of the prosthetic leg 320). For example, the second kinematic data may be received by the control computing device 144 from the shank sensor 136 or 336. For example, the second kinematic data may comprise second orientation data (e.g., a second angle of orientation) for the shank portion 110 of the paretic leg 104 (or the shank section 324 of the prosthetic leg 320). For example, the second angle of orientation for the shank portion 110 (or the shank section 324) may be determined based on the angle between the longitudinal axis β of the shank portion 110 (or the shank section 324) and one of a vertical axis and a horizontal axis. For example, the second kinematic data may further comprise angular velocity data for the shank portion 110 (or the shank section 324), acceleration data for the shank portion 110 (or the shank section 324), or position data for the shank portion 110 (or the shank section 324). For example, the second kinematic data may further comprise muscle activity data for the shank portion 110 of the paretic leg 104. For example, the second kinematic data may also comprise a calculated estimate of the center of mass of the body of the user 102.

At 1030, a limb orientation value may be determined. For example, the limb orientation value may be determined by the control computing device 144 or another computing device. For example, the limb orientation value may be determined based on the first kinematic data and the second kinematic data. For example, the limb orientation value may be determined based on the first angle of orientation (for the thigh portion 108 or thigh section 322) and the second angle of orientation (for the shank portion 110 or the shank section 324). For example, the limb orientation value may be determined based on the formula:
Limb orientation value =atan((sin(Θ)−sin(λ)/(cos(Θ)+cos(λ)));
   where λ=the first angle of orientation and Θ=the second angle of orientation. For example, the formula may represent the basic trigonometry to calculate the relative orientation between the heel of the foot 112, 326 and the pelvis of the paretic leg 104 (or the user of the prosthetic leg 320).

At 1040, a determination may be made that the limb orientation value satisfies an orientation threshold. For example, the determination may be based on the received first kinematic data and the received second kinematic data for the paretic leg 104 (or the prosthetic leg 320). The determination may be made by the control computing device 144 or another computing device. For example, the control computing device 144 may compare the limb orientation value to the orientation threshold to determine if the limb orientation value satisfies the orientation threshold. The limb orientation value satisfies the orientation threshold if the difference is one of less than or less than or equal to the orientation threshold. In other examples, termination or reduction of assistance to the paretic leg 104 (or the prosthetic leg 320) may be based on the limb orientation value not satisfying the threshold and the control computing device 144 may determine that the limb orientation value does not satisfy the orientation threshold based on the limb orientation value being less than or less than or equal to the orientation threshold. For example, the orientation threshold may be anywhere in the range of about 0 degrees to about −10 degrees.

At 1050, the provision of at least one of electrical stimuli or motorized assistance may be terminated or reduced at the paretic leg 104 (or the prosthetic leg 320). Based on the control computing device 144 determining that the user 102 is likely transitioning to a quiet stance phase, all electrical or motorized assistance may be terminated or reduced, as it may not be needed by the user 102 (or is needed to a lesser extent) when in a quiet stance (e.g., standing still). For example, the control computing device 144 or another computing device may cause the electrical stimuli and/or the motorized assistance to be terminated or reduced to the paretic leg 104 (or the prosthetic leg 320). Determining whether to terminate or reduce one or both of electrical stimuli or motorized assistance may be based on whether one or both are being provided to the paretic leg 104 (or the prosthetic leg 320) of the user 102. For example, if only motorized assistance is being provided to the paretic leg 104 (or the prosthetic leg 320) of the user 102, then only motorized assistance needs to be terminated or reduced. Likewise if only electrical stimuli is being provided to the paretic leg 104 (or the remaining portion of the thigh of the amputated leg), only electrical stimuli needs to be terminated or reduced.

For example, the control computing device 144 may send a signal to one or more of the motors 128, 152 or 328, 352 to terminate or reduce motorized assistance and/or to the electrodes 130A-B, 132 or the pulse generator 202 to terminate providing or reduce the provision of electrical stimuli to the paretic leg 104 (or the prosthetic leg 320). For example, the electrical stimuli and/or motorized assistance may be terminated or reduced based on the limb orientation value satisfying the orientation threshold. For example, the electrical stimuli and/or motorized assistance may be terminated or reduced based on the time it took to complete a current phase of the gait cycle satisfying a duration threshold as described in 1110-1160 of FIG. 11 below.

For example, one or more of the motors 128, 152 or 328, 352 may receive the signal from the control computing device 144 and may terminate or reduce motorized assistance. For example, the electrodes 130A-B, 132 or the pulse generator 202 may receive the signal from the control computing device 144 and may terminate or reduce electrical stimuli to all portions of the paretic leg 104 (or the remaining portion of the thigh of the amputated leg).

The method 1000 may comprise the control computing device 144 determining, based on the received first kinematic data, the angular velocity of the thigh portion 108 of the paretic leg (or the thigh section 322 of the prosthetic leg 320). The control computing device 144 may compare the angular velocity of the thigh portion 108 (or the thigh section 322) to an angular velocity threshold to determine if the angular velocity of the thigh portion 108 (or the thigh section 322) satisfies the angular velocity threshold. For example, the control computing device 144 may determine that the angular velocity of the thigh portion 108 (or the thigh section 322) satisfies the angular velocity threshold if the angular velocity is one of less than or less than or equal to the angular velocity threshold. In other examples, termination or reduction of assistance to the paretic leg 104 (or the prosthetic leg 320) may be based on the angular velocity not satisfying the angular velocity threshold and the control computing device 144 may determine that the angular velocity does not satisfy the angular velocity threshold based on the angular velocity being less than or less than or equal to the angular velocity threshold. For example, terminating or reducing the motorized assistance and/or electrical stimuli may be further based on the angular velocity of the thigh portion 108 satisfying the angular velocity threshold. For example, the angular velocity threshold may be anywhere in the range of about 0 degrees per second to about 100 degrees per second.

FIG. 11 shows an example method 1100 for terminating or reducing assistance for leg movement. Referring to FIGS. 1-3 and 11, the method 1100 may be completed by one or more of the control computing device 144, the brace 120 (or prosthetic leg 320), and/or the pulse generator 202. At 1110, one or more duration thresholds for one or more phases of a gait motion for a paretic leg 104 (or the prosthetic leg 320) of the user 102 may be determined. For example, the one or more duration thresholds may be determined by control computing device 144, the brace 120 (or prosthetic leg 320), or the pulse generator 102. For example, the one or more duration thresholds may be input into the control computing device 144. For example, the one or more duration thresholds may be determined by the control computing device 144 based on historical gait data for the paretic leg 104 (or the prosthetic leg 320) of the user 102. In certain examples, a duration threshold may be determined for each phase of a gait motion for the paretic leg 104 (or the prosthetic leg 320) of the user 102. In certain examples, a duration threshold may be determined for one or certain phases of the gait motion. For example, the duration threshold may be determined for the loading response or midstance phases of the gait motion for the paretic leg 104 (or the prosthetic leg 320) of the user 102. For example, the duration thresholds may be standardized or may be particular to each individual user 102.

For example, the control computing device 144 or another computer may determine and store the duration for one or more phases of a gait motion of the paretic leg 104 (or the prosthetic leg 320) of the user 102 over a predetermined number of cycles or a time period. For example, the control computing device 144 may determine the duration for one or more phases of the gait motion of the paretic leg 104 (or the prosthetic leg 320) for five previous gait cycles. While the example describes determining the duration for five previous gait cycles, this is for example purposes only as the number may be any number greater than zero prior gait cycles.

The control computing device 144 or another computer may determine an average duration for each of the one or more phases of the gait motion. The average duration for each of the one or more phases of the gait cycle may be determined based on the determined duration of each of the one or more phases of the gait cycle from the previous gait cycles. For example, if the number of prior gait cycles was five, the control computing device 144 may sum the duration for the particular phase of the gait cycle for each of the five prior gait cycles and divide that number by five to get the average duration for the particular phase of the gait cycle. In certain examples, the average duration for the particular phase of the gait cycle may be the duration threshold for that particular phase of the gait cycle. In certain examples, the average duration for each particular phase of the gait cycle is adjusted by an adjustment factor to determine the resultant duration threshold for each particular phase of the gait cycle. For example, the adjustment factor may be a value that is added to the average duration or that the average duration is multiplied by. For example, the adjustment factor may have a value greater than 1, such as anywhere within the range of 1.01-5. For example, the control computing device 144 may determine that the average duration for the midstance phase of the gait motion of the paretic leg 104 (or the prosthetic leg 320) for the user 102 is 0.4 seconds. Based on an adjustment factor of 1.5, the control computing device 144 may determine that the duration threshold for the midstance phase is (0.4×1.5), which equals 0.6 seconds. Similarly, based on an adjustment factor of 0.2, the control computing device 144 may determine that the duration threshold for the midstance phase is (0.4+0.2), which equals 0.6 seconds. As discussed above, the duration threshold may be determined for one, multiple, or all phases of the gait motion of the paretic leg 104 (or the prosthetic leg 320) for the user 102.

At 1120, kinematic data may be received. For example, the kinematic data may be associated with the paretic leg 104 (or the prosthetic leg 320). For example, the kinematic data may have been received from one or more sensors (e.g., the sensors 134-138 or 334-338) receiving data about the paretic leg 104 (or the prosthetic leg 320). For example, the kinematic data may be received by the control computing device 144 from one or more of the sensors 134-138 or 334-338. For example, the kinematic data may comprise one or more of velocity data (e.g., angular velocity data) of all or a portion of the paretic leg 104 (or the prosthetic leg 320), acceleration data of all or a portion of the paretic leg 104 (or the prosthetic leg 320), orientation data of all or a portion of the paretic leg 104 (or the prosthetic leg 320), muscle activity data for all or a portion of the paretic leg 104 (or the remaining portion of the thigh of the leg), or position data for all or a portion of the paretic leg 104 (or the prosthetic leg 320). For example, the portion of the paretic leg 104 (or the prosthetic leg 320) may comprise one of the thigh portion 108 of the paretic leg 104 (or the thigh section 322 of the prosthetic leg 320), the shank portion 110 of the paretic leg 104 (or the shank section 324 of the prosthetic leg 320), or the foot 112 of the paretic leg (or the foot section 326 of the prosthetic leg 320). For example, the kinematic data may comprise an orientation (e.g., an angle of orientation) for the thigh portion 108 of the paretic leg 104 (or the thigh section 322 of the prosthetic leg 320). For example, the angle of orientation for the thigh portion 108 (or the thigh section 322) may be determined based on the angle between the longitudinal axis α of the thigh portion 108 (or the thigh section 322) and one of a vertical axis and a horizontal axis. For example, the kinematic data may comprise heel-strike or pressure data from the sensor that detects heel-strike 138 or 338.

At 1130, a current phase of the gait motion for the paretic leg 104 (or the prosthetic leg 320) may be determined. The current phase of the gait motion may be determined by the control computing device 144 or any other computing device. For example, the current phase of the gait motion may be determined based on the received kinematic data for the paretic leg 104 (or the prosthetic leg 320) and/or the calculated estimate of the center of mass of the body of the user 102. For example, the current phase of the gait motion of the paretic leg 104 (or the prosthetic leg 320) may be determined based on orientation of the thigh portion 108 of the paretic leg 104 (or the thigh section 322 of the prosthetic leg 320). For example, the current phase of the gait motion may be determined based on the orientation of the thigh portion 108 (or the thigh section 322) and the orientation of the shank portion 110 (or the shank section 324) of the paretic leg 104 (or the prosthetic leg 320). For example, the current phase of the gait motion may be determined based on the heel-strike or pressure data from the heel-strike sensor 138 or 338. For example, the current phase of the gait motion may be determined based on the heel-strike or pressure data from the heel strike sensor 138 or 338, the orientation, muscle activity, or position data for the thigh portion 108 or thigh section 322 from the thigh sensor 134 or 338, and/or the orientation, muscle activity, or position data for the shank portion 110 from the shank sensor 136 or 336. For example, the current phase of the gait motion of the paretic leg 104 (or the prosthetic leg 320) may be further determined based on the velocity data (e.g., angular velocity data) for the thigh portion 108 (or the thigh section 322) and/or the velocity data (e.g., angular velocity data) for the shank portion 110 (or the shank section 324) of the paretic leg 104 (or the prosthetic leg 320). For example, the current phase of the gait motion of the paretic leg 104 (or the prosthetic leg 320) may further be determined based on the calculated estimate of the center of mass of the body of the user 102. For example, the current phase of the gait motion for the paretic leg 104 (or the prosthetic leg 320) may be one of heel-strike, foot flat, midstance, heel off, toe off, swing phase (e.g., initial swing, mid-swing, or terminal swing) or stance.

At 1140, a time the paretic leg 104 (or the prosthetic leg 320) is in the current phase of the gait motion may be determined. The time the paretic leg 104 (or the prosthetic leg 320) is in the current phase of the gait motion may be determined by the control computing device 144 or any other computing device. For example, the control computing device, based on determining the phases of the gait motion at 1130, may determine transition points between each phase of the gait motion. Based on a transition point from one phase of the gait motion/cycle to another phase of the gait cycle occurring, the control computing device 144 may begin a timer to determine how long the user is in the particular phase of the gait cycle. The control computing device 144 may stop the timer and begin another timer (or reset the original timer) when the paretic leg 104 gets to another transition point from one phase to the next phase of the gait cycle for the paretic leg 104 (or the prosthetic leg 320). The control computing device 144 may continuously or periodically compare the value of the time for the particular phase of the gait cycle to the duration threshold for that phase of the gate cycle to determine if the value of the time that the user has already taken to move through the particular phase of the gate cycle satisfies (e.g., is greater than or greater than or equal to) the duration threshold for that phase of the gait cycle. For example, the control computing device 144 may determine, based on the timer value, the paretic leg 104 has already taken more than 0.6 seconds (e.g., 0.7 seconds) in the midstance or loading response phase of the gait cycle.

At 1150, a determination may be made that the time to complete a particular phase of the gait cycle for the paretic leg 104 (or the prosthetic leg 320) of the user 102 satisfies (e.g., is greater than or greater than or equal to) a duration threshold for that phase of the gait cycle. For example, the determination may be based on the timer value for the current phase of the gait cycle and the determined duration threshold. The determination the duration threshold has been satisfied may be made by the control computing device 144 or another computing device. For example, the control computing device 144 may compare the timer value for the current phase of the gait cycle for the paretic leg 104 (or the prosthetic leg 320) of the user 102 to the duration threshold for that particular phase of the gait cycle to determine if the timer value for the current phase satisfies the duration threshold. For example, the timer value for the current phase satisfies the duration threshold if the timer value is one of greater than or greater than or equal to the duration threshold. In other examples, termination or reduction of assistance to the paretic leg 104 (or the prosthetic leg 320) may be based on the timer value for the current phase not satisfying the threshold and the control computing device 144 may determine that the timer value for the current phase does not satisfy the duration threshold for that phase based on the timer value being greater than or greater than or equal to the duration threshold for that phase.

At 1160, the provision of at least one of electrical stimuli or motorized assistance may be terminated or reduced at the paretic leg 104 (or the prosthetic leg 320) or the provision may be modified to move the paretic leg (or the prosthetic leg 320) towards a quiet stance phase. Based on the control computing device 144 determining one of that the user 102 is likely transitioning to a quiet stance phase or the system is not sure if one or more phases of the gait cycle for the paretic leg 104 (or the prosthetic leg 320) were missed during the evaluation of the kinematic data, all electrical and/or motorized assistance may be terminated or electrical and/or motorized assistance may be modified to begin moving the paretic leg 104 (or the prosthetic leg 320) towards the quiet stance phase. The termination or modification of the electrical stimuli and/or motorized assistance may occur because it is not needed by the user 102 (or is needed to a lesser extent) when in or moving towards the quiet stance (e.g., standing still) phase or may be detrimental the user 102, as it may be provided to the wrong portions of the leg 104 (or the prosthetic leg 320) or at the wrong time or in the wrong direction based on the correct phase that the paretic leg 104 (or the prosthetic leg 320) is currently in. For example, the provision of at least one of electrical stimuli or motorized assistance may be terminated or modified at the paretic leg 104 (or the prosthetic leg 320) based on the time value for the paretic leg 104 (or the prosthetic leg 320) in the current phase of the gait cycle satisfying the duration threshold for that phase of the cycle. For example, the control computing device 144 may compare the time the paretic leg (or the prosthetic leg 320) has been in the midstance or loading response phase of 0.7 seconds to the duration threshold for the midstance or loading response phase of 0.6 and determine that the time value for the paretic leg 104 (or the prosthetic leg 320) in the midstance or loading response phase satisfies the duration threshold. For example, the time value being greater than or greater than or equal to the duration threshold may be an indicator that the user is slowing down the gait cycle speed to come to a stop (e.g., quiet stance) or that a transition from the current phase of the gait cycle to the next phase of the gait cycle was missed based on the analysis of the kinematic data at 1120-1130. For example, if the time value for the paretic leg 104 (or the prosthetic leg 320) in the midstance phase reached 1.8 seconds (as compared to a duration threshold of 0.6 seconds), or any other value above the duration threshold, it may indicate that one or more subsequent phases of the gait cycle for the paretic leg 104 (or the prosthetic leg 320) were missed or misidentified and continued provision of motorized assistance or electrical stimuli, at the current level or at any level, may not be beneficial to the user 102.

For example, the control computing device 144 or another computing device may cause the electrical stimuli and/or the motorized assistance to be terminated or modified to the paretic leg 104 (or the prosthetic leg 320). Determining whether to terminate or modify one or both of electrical stimuli or motorized assistance may be based on whether one or both are being provided to the paretic leg 104 (or the prosthetic leg 320) of the user 102. For example, if only motorized assistance is being provided to the paretic leg 104 (or the prosthetic leg 320) of the user 102, then only motorized assistance needs to be terminated or modified. Likewise if only electrical stimuli is being provided to the paretic leg 104 (or the remaining portion of thigh of the amputated leg), only electrical stimuli needs to be terminated or modified.

For example, the control computing device 144 may send a signal to one or more of the motors 128, 152 or 328, 352 to terminate or modify motorized assistance and/or to the electrodes 130A-B, 132 or the pulse generator 202 to terminate providing or modify the provision of electrical stimuli to the paretic leg 104 (or the prosthetic leg 320) to begin moving the paretic leg (or the prosthetic leg 320) from the current phase of the gait cycle towards the quiet stance phase of the gait cycle.

For example, one or more of the motors 128, 152 or 328, 352 may receive the signal from the control computing device 144 and may terminate or modify motorized assistance to begin moving the paretic leg (or the prosthetic leg 320) from the current phase of the gait cycle towards the quiet stance phase of the gait cycle. For example, the electrodes 130A-B, 132 or the pulse generator 202 may receive the signal from the control computing device 144 and may terminate or modify electrical stimuli to all or portions of the paretic leg 104 (or the remaining portion of thigh of the amputated leg) to begin moving the paretic leg (or the prosthetic leg 320) from the current phase of the gait cycle towards the quiet stance phase of the gait cycle ..

FIG. 12 shows a system 1200 for providing or terminating assistance for leg movement. The control computing device 144 or another computing device may be a computer 1201 as shown in FIG. 12.

The computer 1201 may comprise one or more processors 1203, a system memory 1213, and a bus 1214 that couples various components of the computer 1201 including the one or more processors 1203 to the system memory 1213. In the case of multiple processors 1203, the computer 1201 may utilize parallel computing.

The bus 1214 may comprise one or more of several possible types of bus structures, such as a memory bus, memory controller, a peripheral bus, an accelerated graphics port, and a processor or local bus using any of a variety of bus architectures.

The computer 1201 may operate on and/or comprise a variety of computer-readable media (e.g., non-transitory). Computer-readable media may be any available media that is accessible by the computer 1201 and includes, non-transitory, volatile and/or non-volatile media, and removable and non-removable media. The system memory 1213 has computer-readable media in the form of volatile memory, such as random access memory (RAM), and/or non-volatile memory, such as read-only memory (ROM). The system memory 1213 may store data and/or program modules such as an operating system 1205, the gait detection engine 1206, and sensor metrics 1207 that are accessible to and/or are operated on by the one or more processors 1203.

The computer 1201 may also comprise other removable/non-removable, volatile/non-volatile computer storage media. The mass storage device 1204 may provide non-volatile storage of computer code, computer-readable instructions, data structures, program modules, and other data for the computer 1201. The mass storage device 1204 may be a hard disk, a removable magnetic disk, a removable optical disk, magnetic cassettes or other magnetic storage devices, flash memory cards, CD-ROM, digital versatile disks (DVD) or other optical storage, RAM, ROM, electrically erasable programmable read-only memory (EEPROM), and the like.

Any number of program modules may be stored on the mass storage device 1204. An operating system 1205, the gait detection engine 1206, and sensor metrics 1207 may be stored on the mass storage device 1204. One or more of the operating system 1205, gait detection engine 1206, and sensor metrics 1207 (or some combination thereof) may comprise one or more program modules.

A user may enter commands and information into the computer 1201 via an input device, such as the control computing module 144. Such input devices include, but are not limited to, a keyboard, pointing device (e.g., a computer mouse or remote control), a microphone, a joystick, a scanner, tactile input devices such as gloves, and other body coverings, a motion sensor, the control computing module 144, and the like These and other input devices may be connected to the one or more processors 1203 via a human-machine interface 1202 that is coupled to the bus 1214, but may be connected by other interface and bus structures, such as a parallel port, game port, an IEEE 1394 Port (also known as a Firewire port), a serial port, network adapter 1209, and/or a universal serial bus (USB).

A display device 1212 may also be connected to the bus 1214 via an interface, such as a display adapter 1210. It is contemplated that the computer 1201 may have zero displays or more than one display adapter 1210 and the computer 1201 may have more than one display device 1212. A display device 1212 may be a monitor, an LCD (Liquid Crystal Display), a light-emitting diode (LED) display, a television, smart lens, smart glass, and/or a projector. In addition to the display device 1212, other output peripheral devices may comprise components such as speakers (not shown) and a printer (not shown) which may be connected to the computer 1201 via Input/Output Interface 1211. Any step and/or result of the methods may be output (or caused to be output) in any form to an output device. Such output may be any form of visual representation, including, but not limited to, textual, graphical, animation, audio, tactile, and the like. The display 1212 and computer 1201 may be part of one device, or separate devices.

The computer 1201 may operate in a networked environment using logical connections to one or more other devices, such as the one or more sensors 1216, one or more motors and/or sensors 1218, and/or a pulse generator 1220. The one or more sensors 1216 may comprise the sensors 134-142, 154 of FIG. 1 or 334-338, 354 of FIG. 3. The one or more motors and/or sensors 1218 may comprise the motors 128, 152 or 328, 352. The pulse generator 1220 may comprise the pulse generator 202 of FIG. 2. Logical connections between the computer 1201, the one or more sensors 1216, the motor and/or sensor 1218, and the pulse generator 1220 may be made via a network 1215, such as a local area network (LAN) and/or a general wide area network (WAN) and one or more network devices (e.g., a router, an edge device, an access point or other common network nodes, such as a gateway). Such network connections may be through a network adapter 1209. The network adapter 1209 may be implemented in both wired and wireless environments. Such networking environments are conventional and commonplace in dwellings, offices, enterprise-wide computer networks, intranets, and the Internet.

Application programs and other executable program components such as the operating system 1205, the gait detection engine 1206, and the sensor metrics 1207 are shown herein as discrete blocks, although it is recognized that such programs and components may reside at various times in different storage components of the computing device 1201, and are executed by the one or more processors 1203 of the computer 1201. Any of the disclosed methods may be performed by processor-executable instructions embodied on computer-readable media.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

1. A method comprising:
receiving, by a computing device, kinematic data associated with one of a paretic leg or a prosthetic leg;
determining, based on the kinematic data, a current phase of a gait motion for the one of the paretic leg or the prosthetic leg; and
providing, based on the current phase of the gait motion for the one of the paretic leg or the prosthetic leg, at least one of electrical stimuli or motorized assistance to the one of the paretic leg or the prosthetic leg.

2. The method of claim 1, wherein determining the current phase of the gait motion for the one of the paretic leg or the prosthetic leg comprises determining the one of the paretic leg or the prosthetic leg is in a swing phase of the gait motion.

3. The method of claim 1, wherein determining the current phase of the gait motion for the one of the paretic leg or the prosthetic leg comprises determining the one of the paretic leg or the prosthetic leg is in a stance phase of the gait motion.

4. The method of claim 1, wherein the kinematic data comprises one or more of a position of the one of the paretic leg or the prosthetic leg, an angular velocity of the one of the paretic leg or the prosthetic leg, an orientation of the one of the paretic leg or the prosthetic leg, or an acceleration of the one of the paretic leg or the prosthetic leg.

5. The method of claim 1, wherein the kinematic data is for a portion of the one of the paretic leg or the prosthetic leg.

6. (canceled)

7. The method of claim 1, wherein the kinematic data comprises an angle of orientation of a thigh portion of the paretic leg relative to a vertical axis.

8. The method of claim 1, wherein the kinematic data comprises an angle of orientation of a thigh section of the prosthetic leg relative to a vertical axis.

9. A method comprising:
receiving kinematic data for a portion of one of a paretic leg or a prosthetic leg of a user;
determining, based on the kinematic data, an orientation of the portion of the one of the paretic leg or the prosthetic leg; and
initiating, based on the orientation of the portion of the one of the paretic leg or the prosthetic leg at least one of electrical stimuli or motorized assistance for at least the portion of the one of the paretic leg or the prosthetic leg.

10. The method of claim 9, further comprising:
determining, based on the kinematic data, an angular velocity for the portion of the one of the paretic leg or the prosthetic leg; and
determining the angular velocity satisfies an angular velocity threshold,
wherein initiating at least one of the electrical stimuli or the motorized assistance is further based on the angular velocity satisfying the angular velocity threshold.

11. The method of claim 9, wherein the kinematic data comprises one or more of a position of the portion of the one of the paretic leg or the prosthetic leg, an angular velocity of the portion of the one of the paretic leg or the prosthetic leg, the orientation of the portion of the one of the paretic leg or the prosthetic leg, or an acceleration of the portion of the one of the paretic leg or the prosthetic leg.

12. The method of claim 9, wherein the portion of the one of the paretic leg or the prosthetic leg comprises a thigh portion of the user or a thigh section of the prosthetic leg.

13. The method of claim 9, wherein the kinematic data comprises an angle of orientation of one of a thigh portion of the paretic leg or a thigh portion of the prosthetic leg of the user.

14. (canceled)

15. The method of claim 9, wherein the orientation of the portion of the one of the paretic leg or the prosthetic leg satisfies a threshold, wherein the threshold comprises an angle relative to a vertical axis.

16. A method comprising:
receiving first kinematic data or heel-strike data indicative of foot-floor contact for a portion of a non-paretic leg of a user;
receiving second kinematic data for a portion of one of a paretic leg or a prosthetic leg of the user;
determining based on the second kinematic data, an orientation of the portion of the one of the paretic leg or the prosthetic leg satisfies a threshold; and
terminating or reducing, based on the first kinematic data or the heel-strike data for the portion of the non-paretic leg indicating the foot-floor contact for the portion of the non-paretic leg and the orientation of the portion of the one of the paretic leg or the prosthetic leg satisfying the threshold, at least one of supply of electrical stimuli or motorized assistance to at least the portion of the one of the paretic leg or the prosthetic leg of the user.

17. (canceled)

18. The method of claim 16, wherein the first kinematic data or the heel-strike data indicates a portion of a foot of the non-paretic leg contacting a floor surface.

19. The method of claim 16, wherein each of the first kinematic data and the second kinematic data comprises one or more of a position of the portion of the one of the paretic leg or the prosthetic leg, an angular velocity of the portion of the one of the paretic leg or the prosthetic leg, the orientation of the portion of the one of the paretic leg or the prosthetic leg, or an acceleration of the portion of the one of the paretic leg or the prosthetic leg.

20. The method of claim 16, wherein the second kinematic data comprises an orientation of at least one of a thigh portion or a shank portion of the one of the paretic leg or the prosthetic leg.

21-54. (canceled)

55. The method of claim 1, further comprising determining, based on the kinematic data an orientation of a portion of the one of the paretic leg or the prosthetic leg, wherein providing the at least one of the electrical stimuli or the motorized assistance is further based on the orientation of the portion of the one of the paretic leg or the prosthetic leg.

56. The method of claim 9, further comprising determining, based on the kinematic data, a current phase of a gait motion for the one of the paretic leg or the prosthetic leg, wherein initiating the at least one of the electrical stimuli or the motorized assistance is further based on the current phase of the gait motion for the one of the paretic leg or the prosthetic leg.

57. The method of claim 16, further comprising:
receiving third kinematic data for the portion of one of the paretic leg or the prosthetic leg;
determining, based on the third kinematic data, at least one of a second orientation of the portion of the one of the paretic leg or the prosthetic leg or a current phase of a gait motion for the one of the paretic leg or the prosthetic leg; and
providing, based on the at least one of the second orientation or the current phase of the gait motion, at least one of the electrical stimuli or the motorized assistance to the one of the paretic leg or the prosthetic leg.
